(19) United States
(12) Patent Application Publication
(10) Pub. No.: US 2006/0122127 A1
(43) Pub. Date: Jun. 8, 2006

(54) Methods for reducing the side effects associated with mirtzapine treatment

(75) Inventors: Srinivas Rao, Encinitas, CA (US); Jay D. Kranzler, La Jolla, CA (US); Jeffrey J. Anderson, San Diego, CA (US)

Correspondence Address: WILSON SONSINI GOODRICH & ROSATI, 650 PAGE MILL ROAD, PALO ALTO, CA, 94304-1050, US

(73) Assignee: Cypress Bioscience, Inc.

(21) Appl. No.: 11/282,004

(22) Filed: Nov. 17, 2005

Related U.S. Application Data
(60) Provisional application No. 60/628,823, filed on Nov. 17, 2004.
(60) Provisional application No. 60/635,586, filed on Dec. 13, 2004.
(60) Provisional application No. 60/659,652, filed on Mar. 8, 2005.

(51) Int. Cl. A61K 31/7008; A61K 31/551; A61K 31/137; A61K 31/42; A61K 31/13; A61K 31/425

(52) U.S. Cl. 514/23; 514/221; 514/649; 514/662; 514/379; 514/373

(57) ABSTRACT

Compositions, and methods of use thereof, are provided for the prevention or treatment of side effects associated with the use of drugs that act as 5HT2/5HT3 serotonin receptor antagonists and alpha-2 adrenergic receptor antagonists (5HT2/5HT3 antagonist/alpha-2 antagonist). The method involves using dopamine-releasing compounds, such as amantadine, anticonvulsants, such as zonisamide, or dopamine/norepinephrine reuptake inhibitors, such as bupropion, in combination with 5HT2/5HT3 antagonist/alpha-2 antagonists, such as mirtazapine, to reduce the excessive daytime drowsiness and/or weight gain associated with 5HT2/5HT3 antagonist/alpha-2 antagonist use for the treatment of disorders, such as, depression, schizophrenia, anxiety disorders, sleep-related breathing disorders, insomnia, migraine headache, chronic tension-type headache, hot flashes, lower back pain, neuropathic pain and functional somatic syndromes. Formulations of dopamine-releasing compounds or anticonvulsants with 5HT2/5HT3 antagonist/alpha-2 antagonists are provided. In particular embodiments, combination therapy with mirtazapine and zonisamide provides relief from chronic low back pain, while reducing or avoiding side effects associated with monotherapy with mirtazapine or zonisamide.

### CROSS-REFERENCE

This application claims the benefit of U.S. Provisional Application No. 60/628,823, filed Nov. 17, 2004; U.S. Provisional Application No. 60/635,586, filed Dec. 13, 2004; and U.S. Provisional Application No. 60/659,652, filed Mar. 8, 2005. Each of these provisional applications is expressly incorporated herein in its entirety.

### FIELD OF THE INVENTION

This invention generally relates to methods and compositions for the pharmacological treatment or alleviation of the side effects associated with the use of mirtazapine in the treatment of a disorder.

### BACKGROUND OF THE INVENTION

Mirtazapine has been utilized effectively in the treatment of depression. It is also effective in the treatment of schizophrenia, anxiety disorders, sleep apnea, insomnia, migraine headache, chronic tension-type headache, hot flashes, and fibromyalgia. Mirtazapine owes its diverse utility in treating this range of disorders to its diverse pharmacology. Mirtazapine acts as an antagonist at presynaptic alpha-2 adrenergic receptors on both norepinephrine and serotonin (5-HT) presynaptic nerve terminals. In addition, it acts as a potent antagonist at 5HT2A serotonin receptors, 5HT2C serotonin receptors, 5HT3 serotonin receptors, and histamine H1 receptors. Mirtazapine is a very weak inhibitor of norepinephrine reuptake and alpha-1 adrenergic receptors, and has no effect on the reuptake of dopamine or 5-HT. The net outcome of these effects is increased noradrenergic and serotonergic activity, especially at 5HT1A serotonin receptors. However, Mirtazapine can produce side effects which lead to reduced efficacy and result in patients being taken off of the medication. The side effects include marked gains in body weight and excessive daytime sleepiness or drowsiness. The weight gain is likely due to the 5HT2C and H1 receptor antagonistic effects of mirtazapine, while the excessive daytime drowsiness is likely a result of H1 receptor blockade.

The rates of obese and overweight people have increased drastically over the last decade and there is a high prevalence of obesity in patients with mental illness. Hence, highly effective drugs like mirtazapine, which produce increases in appetite and body weight, may be too high risk for use in this patient population. Similarly, the excessive daytime drowsiness produced by mirtazapine can negatively impact driving and job performance. To reduce the propensity for drowsiness, mirtazapine is often administered at night. However, because of the long elimination T1/2 (20-40 h) of this drug, drowsiness often occurs even the day following administration. Having a means to reduce these side effects would greatly enhance the effectiveness of mirtazapine pharmacotherapy.

Chronic low back pain (CLBP) is a common musculoskeletal disorder that is characterized by pain in the lower back lasting at least 3 months. While a small subset of these patients have existing structural abnormalities or tissue injury, in 90% of CLBP patients the disorder has an unknown etiology. CLBP affects at least 10-15% of the adult population and incurs approximately $50 billion in health care costs, disability claims, and lost productivity. Currently available drug therapies for CLBP typically provide only marginal or short term benefit and have dose-limiting tolerability issues. For most patients there are few effective treatment alternatives, and complete relief is rare. Such treatments as exist tend to be characterized by poor efficacy, treatment-limiting side effects, or both. For example, it would be desirable to use mirtazapine for the treatment of CLBP; however, the side-effects of mirtazapine, including excessive daytime sleepiness, sedation and weight gain have heretofore rendered such treatment impractical.

There is thus a need for compositions and methods of treating or alleviating the side effects associated with mirtazapine for use in the treatment of disorders, such as, depression, schizophrenia, anxiety disorders, sleep-related breathing disorders, snoring, insomnia, migraine headache, chronic tension-type headache, hot flashes, chronic lower back pain, neuropathic pain and functional somatic syndromes. In particular, there is a need for methods of treating chronic lower back pain with mirtazapine, wherein the side effects of standard mirtazapine treatment are reduced or eliminated.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

The foregoing and other needs are met by embodiments of the present invention, which provide methods of treating chronic lower back pain, comprising co-administering to a patient suffering from CLBP a therapeutically effective amount of mirtazapine and zonisamide. The combination of zonisamide with mirtazapine is effective to reduce one or more side-effects of mirtazapine, such as excessive daytime sleepiness, sedation and weight gain. Combination may be in a single dosage form, in separate dosage forms administered at substantially the same time or in separate dosage forms administered as part of the same treatment regime but at different times during the day.

The foregoing and further needs are more generally met by compositions, and methods of use thereof for the treatment or alleviation of side effects associated with the use of drugs that act as 5HT2/5HT3 serotonin receptor antagonists and alpha-2 adrenergic receptor antagonists (5HT2/5HT3 antagonist/alpha-2 antagonist).

The foregoing and other needs are further met by embodiments that provide dopamine-releasing compounds, such as amantadine, anticonvulsants, such as zonisamide, or dopamine/norepinephrine reuptake inhibitors, such as bupropion, in combination with 5HT2/5HT3 antagonist/alpha-2 antagonists, such as mirtazapine, to reduce the excessive daytime drowsiness and/or weight gain associated with 5HT2/5HT3 antagonist/alpha-2 antagonist use for the treatment of disorders such as depression, schizophrenia, anxiety disorders, sleep-related breathing disorders, snoring, insomnia, migraine headache, chronic tension-type headache, hot flashes, lower back pain, neuropathic pain and functional somatic syndromes, including Chronic Fatigue Syndrome (CFS), Fibromyalgia Syndrome (FMS), and Irritable Bowel Syndrome (IBS). The dopamine-releasing compounds, anticonvulsants, or dopamine/norepinephrine reuptake inhibitors may be administered either simultaneously with the 5HT2/5HT3 antagonist/alpha-2 antagonist or after the latter drug is administered. The dopamine-releasing compound, anticonvulsant or dopamine/norepinephrine reuptake inhibitor may also improve the efficacy of the 5HT2/5HT3 antagonist/alpha-2 antagonist in the treatment of a particular disorder.

The foregoing and other needs are further met by embodiments of the invention, which provide formulations of dopamine-releasing compounds, anticonvulsants, or dopamine/norepinephrine reuptake inhibitors with 5HT2/5HT3 antagonist/alpha-2 antagonists. In additional embodiments, the formulations allow for immediate release of the 5HT2/5HT3 antagonist/alpha-2 antagonists and delayed release of the dopamine-releasing compounds, anticonvulsants, or dopamine/norepinephrine reuptake inhibitors.

### DETAILED DESCRIPTION OF THE INVENTION

The present invention relates to the reduction of side effects associated with a 5HT2/5HT3 antagonist/alpha-2 antagonist in the treatment of chronic lower back pain, depression, schizophrenia, anxiety disorders, sleep apnea, snoring, insomnia, migraine headache, chronic tension-type headache, hot flashes, and functional somatic syndromes include an effective amount of a dopamine-releasing compound, anticonvulsant or dopamine/norepinephrine reuptake inhibitor in combination with the 5HT2/5HT3 antagonist/alpha-2 antagonist. The 5HT2/5HT3 antagonist/alpha-2 antagonist and the dopamine-releasing compound, anticonvulsant or dopamine/norepinephrine reuptake inhibitor may be administered in the same or different dosage forms and may be administered at substantially the same time or at different times during the day.

In certain embodiments, the invention relates to reduction of the side effects of mirtazapine in the treatment of chronic low back pain, in which zonisamide is co-administered with mirtazapine or setiptiline in the same treatment milieu. In particular embodiments, mirtazapine or setiptiline may be administered at night, before the patient goes to sleep, while zonisamide is administered in the morning or at some other time during the day. In other embodiments, zonisamide is administered with mirtazapine or setiptiline, either in the same dosage form or in separate dosage forms but at substantially the same time. In further embodiments, zonisamide may be administered at some time during the day, while mirtazapine or setiptiline is administered at night.

I. Compositions

Compositions with reduced side effects associated with the use of a 5HT2/5HT3 antagonist/alpha-2 antagonist in the treatment of depression, schizophrenia, anxiety disorders, sleep apnea, snoring, insomnia, migraine headache, chronic tension-type headache, hot flashes, and functional somatic syndromes include an effective amount of a dopamine-releasing compound, anticonvulsant or dopamine/norepinephrine reuptake inhibitor in combination with the 5HT2/5HT3 antagonist/alpha-2 antagonist. The combination may also improve the efficacy of the 5HT2/5HT3 antagonist/alpha-2 antagonist in the treatment of certain disorders.

A. Drugs with 5HT2/5HT3 Serotonin Receptor Antagonist and Alpha-2 Adrenergic Receptor Antagonist Activity

Useful drugs include compounds that act as antagonists at both the 5HT2 and 5HT3 serotonin receptors and at alpha-2 adrenergic receptors (5HT2/5HT3 antagonist/alpha-2 antagonists). In some embodiments of the invention, such compounds are mirtazapine (1,2,3,4,10,14b-hexahydro-2-methylpyrazino[2,1-a]pyrido [2,3-c]benzazepine) and setiptiline (1,2,3,4-tetrahydro-2-methyl-9H-dibenzo [3,4:6,7] cyclohepta [1,2-C] pyridine maleate).

1. Mirtazapine

Mirtazapine is currently approved in multiple countries for the treatment of depression; the first approval occurred in 1994.

Mirtazapine's chemical name is 1,2,3,4,10,14b-hexahydro-2-methylpyrazino [2,1-a]pyrido [2,3-c]benzazepine; the chemical structure is as follows: 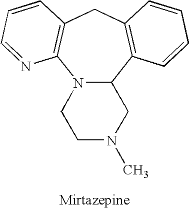
As is clear from the structure, mirtazapine is a chiral compound, and only the racemate has been commercialized to date. Nonetheless, reference to mirtazapine, unless otherwise modified herein, embraces the racemate and the enantiomers, as well as pharmaceutically acceptable salts and polymorphs thereof.

The mechanism by which mirtazapine exerts its antidepressant effects is not fully understood, a situation that is consistent with other drugs approved for use for depression. Pharmacologically, mirtazapine enhances central noradrenergic and serotonergic activity. However, the agent has minimal effects upon peripheral serotonin levels, thus minimizing the chance for serotonin syndrome when used in combination with SSRI or TCA antidepressants. Studies have shown that mirtazapine acts as an antagonist at central presynaptic (alpha)₂ adrenergic inhibitory autoreceptors and heteroreceptors, an action that is postulated to result in an increase in central noradrenergic and serotonergic activity. Mirtazapine is a potent antagonist of 5-HT₂ and 5-HT₃ receptors, but lacks significant affinity for the 5-HT_{1A} and 5-HT_{1B} receptors. Mirtazapine is a potent antagonist of histamine (H₁) receptors, a property that may explain its prominent sedative effects. Mirtazapine is a moderate peripheral (alpha), adrenergic antagonist, a property that may explain the occasional orthostatic hypotension reported in association with its use. Mirtazapine is a moderate antagonist at muscarinic receptors, a property that may explain the relatively low incidence of anti-cholinergic side effects associated with its use.

Mirtazapine: Experience in Chronic Pain

Mirtazapine is a potent antagonist of central 5HT₂, 5HT₃ and α₂ receptors. Mirtazapine stimulates both norepinephrine- and serotonin-mediated neurotransmission by blocking presynaptic α₂ receptors, which enhances norepinephrine release, and by antagonizing α₂ heteroreceptors on serotonin neurons, which increases serotonin release. Thus, mirtazapine is a desirable analgesic for the treatment of chronic lower back pain. Nonetheless, heretofore, the use of mirtazapine of chronic lower back pain has been limited by the sedative effects of mirtazapine, which can persist for some time after administration of the drug. Thus, one factor reducing mirtazapine's efficacy in treating chronic lower back pain is excessive daytime sleepiness due to residual sedative effects. Another factor reducing mirtazapine's appeal as an analgesic is that it tends to induce weight gain in patients over time.

Setiptiline is a drug having antagonist activity toward the central 5HT₂, 5HT₃ and α₂ receptors and possesses indications and pharmacology that are very similar to those of mirtazapine. It is thus an aspect of the invention that all or part of the mirtazapine may be replaced by an equipotent (on a monotherapeutic basis) amount of setiptiline. The potency of setiptiline as compared to that of mirtazapine is considered within the skill of the ordinary clinician.

B. Drugs Used in Combination with 5HT2/5HT3 Antagonist/Alpha-2 Antagonists

1. Dopamine-Releasing Compounds

In some embodiments, dopamine-releasing compounds are used in combination with 5HT2/5HT3 antagonist/alpha-2 antagonists to reduce the excessive daytime drowsiness and/or weight gain associated with 5HT2/5HT3 antagonist/alpha-2 antagonist use for the treatment of the disorders. The dopamine-releasing compounds may also improve the efficacy of the 5HT2/5HT3 antagonist/alpha-2 antagonists.

Useful dopamine-releasing compounds include compounds that induce release of dopamine from pre-synaptic dopaminergic neurons. Preferred compounds include amantadine (1-aminoadamantane hydrochloride), rimantadine (alpha-methytricyclo-(3.3.1.1/0.7)decane-1-methanamine hydrochloride, amphetamines such as methamphetamine (S)-N, (alpha)-dimethylbenzeneethanamine hydrochloride), dextroamphetamine (d-a-methylphenethylamine), and laevoamphetamine, and methylphenidate (methyl a-phenyl-2-piperi-dineeacetate hydrochloride).

In other embodiments, the dopamine-releasing compounds may improve the efficacy of the 5HT2/5HT3 antagonist/alpha-2 antagonists in the treatment of the disorders, especially pain-related disorders. The ability of the dopamine-releasing compounds to release dopamine helps to provide pain relief.

Both racemic and diasteromeric mixtures of the compounds, as well as the individual optical isomers isolated or synthesized, substantially free (more than 90% or 95% pure) of their enantiomeric or diastereomeric partners, may be used. These compounds include racemic rimantadine, (R)-rimantadine, and (S)-rimantadine.

2. Anticonvulsants

In other embodiments, anticonvulsants are used in combination with 5HT2/5HT3 antagonist/alpha-2 antagonists to reduce the excessive daytime drowsiness and/or weight gain associated with 5HT2/5HT3 antagonist/alpha-2 antagonist use for the treatment of the disorders. The anticonvulsants may also improve the efficacy of the 5HT2/5HT3 antagonist/alpha-2 antagonists.

Useful anticonvulsants include compounds that depresses abnormal nerve activity in the brain. Preferred anticonvulsants include barbiturates, including mephobarbital, pentobarbital, and phenobarbital; benzodiazepines, including chlorazepate, clonazepam, and diazepam; GABA analogs, such as gabapentin and tiagabine; hydantoins, such as ethotoin, fosphentyoin, and phenyloin; oxzaolidinediones, such as trimethadione; phenyltriazines, such as lamotrigine; succinimides, such as ethosuximide, methsuximide, phensuximide; and acetazolamide, carbamazepine, felbamate, levetiracetam, oxacarbazepine, primidone, and valproic acid.

In particular embodiments, the anticonvulsant is zonisamide (1,2-benzisoxazole-3-methanesulfonamide) or topiramate (2,3:4,5-Di-O-isopropylidene-β-D-fructopyranose sulfamate), which is a Na⁺ and Ca⁺⁺ channel antagonist that also enhances serotonin neurotransmission through a mechanism different than that of mirtazapine.

Zonisamide (1,2-benzisoxazole-3-methanesulfonamide) is chemically classified as a sulfonamide and unrelated to other anti-seizure agents. The chemical structure is as follows:

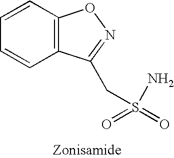

The precise mechanism(s) by which zonisamide exerts its anti-seizure effect is unknown. Zonisamide demonstrated anticonvulsant activity in several experimental models. In animals, zonisamide was effective against tonic extension seizures induced by maximal electroshock but ineffective against clonic seizures induced by subcutaneous pentylenetetrazol. Zonisamide raised the threshold for generalized seizures in the kindled rat model and reduced the duration of cortical focal seizures induced by electrical stimulation of the visual cortex in cats. Furthermore, zonisamide suppressed both interictal spikes and the secondarily generalized seizures produced by cortical application of tungstic acid gel in rats or by cortical freezing in cats. The relevance of these models to human epilepsy is unknown. Zonisamide may produce these effects through action at sodium and calcium channels.

In vitro pharmacological studies suggest that zonisamide blocks sodium channels and reduces voltage-dependent, transient inward currents (T-type Ca2+ currents), consequently stabilizing neuronal membranes and suppressing neuronal hyper-synchronization. In vitro binding studies have demonstrated that zonisamide binds to the GABA/benzodiazepine receptor ionophore complex in an allosteric fashion which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has carbonic anhydrase inhibiting activity, although this pharmacologic effect is not thought to be a major contributing factor in the anti-seizure activity of zonisamide.

In another embodiment, the anticonvulsant may improve the efficacy of the 5HT2/5HT3 antagonist/alpha-2 antagonists in the treatment of the disorders, especially pain-related disorders. The ability of the anticonvulsants to block voltage-dependent sodium and calcium channels as well as enhance the efficiency of GABA binding or activity helps to reduce pain transmission and provide pain relief.

3. Dopamine/Norepinephrine Reuptake Inhibitors

In another embodiment, dopamine/norepinephrine reuptake inhibitors are used in combination with 5HT2/5HT3 antagonist/alpha-2 antagonists to reduce the excessive daytime drowsiness and/or weight gain associated with 5HT2/5HT3 antagonist/alpha-2 antagonist use for the treatment of the disorders. The dopamine/norepinephrine reuptake inhibitors may also improve the efficacy of the 5HT2/5HT3 antagonist/alpha-2 antagonists. Useful compounds include any drug that acts as both a dopamine and a norepinephrine reuptake inhibitor. Preferred dopamine/norepinephrine reuptake inhibitors that may be used include bupropion ((±)-1-(3-chlorophenyl)-2-[(1,1-dimethylethyl)amino]-1-propanone hydrochloride). In addition, sibutramine [N-1-[1-(4-chlorophenyl)cyclobutyl]-3-methylbutyl]-N,N-dimethylamine and its metabolites, didesmethylsibutramine and desmethylsibutramine may be used. Both racemic and diasteromeric mixtures of the compounds, as well as the individual optical isomers isolated or synthesized, substantially free (more than 90% or 95% pure) of their enantiomeric or diastereomeric partners, may be used. These compounds include racemic sibutramine, (+)-sibutramine, (−)-sibutramine, racemic didesmethylsibutramine, (+)-didesmethylsibutramine, (−)-didesmethylsibutramine, racemic desmethylsibutramine, (+)-desmethylsibutramine, and (−)-desmethylsibutramine.

C. Salts, Stereoisomers, Polymorphs and Derivatives

Although described above with reference specific to compounds, one can also utilize stereoisomers, polymorphs, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

Different polymorphs of the compounds may also be used. Polymorphs are, by definition, crystals of the same molecule having different physical properties as a result of the order of the molecules in the crystal lattice. The polymorphic behavior of drugs can be of crucial importance in pharmacy and pharmacology. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity).

Unless otherwise limited herein, recitation of a compound is intended to embrace pharmaceutically acceptable salts, racemates, enantiomers and polymorphs of the compound.

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a patient, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds in vivo. Metabolites include products or intermediates from any metabolic pathway.

D. Formulations

The compounds, or pharmaceutically acceptable salts thereof, or polymorphic variations thereof, can be formulated as pharmaceutical compositions. Such compositions can be administered orally, buccally, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

In some embodiments, formulations combine a dopamine releasing compound, anticonvulsant, or dopamine/norepinephrine reuptake inhibitor with a 5HT2/5HT3 antagonist/alpha-2 antagonist, such as mirtazapine, in a formulation which allows for immediate release of the 5HT2/5HT3 antagonist/alpha-2 antagonist and delayed release of the dopamine-releasing compound, anticonvulsant, or dopamine/norepinephrine reuptake inhibitor. In one embodiment, the dopamine-releasing compound, anticonvulsant, or dopamine/norepinephrine reuptake inhibitor is not released until at least 6 hours after the 5HT2/5HT3 antagonist/alpha-2 antagonist is released. 5HT2/5HT3 antagonist/alpha-2 antagonists, such as mirtazapine, are typically administered once/day at night because of the somnolence they produce. Delayed release of the dopamine-releasing compound, anticonvulsant, or dopamine/norepinephrine reuptake inhibitor is important so that adequate concentrations are available in the circulation following sleep to counteract the excessive daytime sleepiness and/or increased appetite/weight gain associated with 5HT2/5HT3 antagonist/alpha-2 antagonist use.

II. Disorders to be Treated by the 5HT2/5HT3 Antagonist/Alpha-2 Antagonists

A. Chronic Low Back Pain

Chronic low back pain (CLBP) is a common musculoskeletal disorder that is characterized by pain in the lower back lasting at least 3 months. While a small subset of these patients have existing structural abnormalities or tissue injury, in 90% of CLBP patients the disorder has an unknown etiology. CLBP affects at least 10-15% of the adult population and gives rise to approximately $50 billion in health care costs, disability claims, and lost productivity. Existing drug therapies for CLBP typically provide only marginal or short term benefit and have dose-limiting tolerability issues.

Chronic low back pain is defined as pain, muscle tension, or stiffness localized to the lower back persisting for longer than 3 months. About 10% of the cases originate from injuries or degeneration of spinal structures including muscle-ligament injuries, disk herniation, and spinal stenosis. Approximately 90% of cases, however, have no identifiable cause or anatomical abnormalities that clearly explain their symptoms and are designated nonspecific or idiopathic. Manek, N. J. and A. J. MacGregor, Epidemiology of back disorders: prevalence, risk factors, and prognosis. Curr. Opin. Rheumatol., 2005. 17(2): p. 134-40. Nonspecific terms such as strain, sprain, or degenerative processes are commonly used. Diagnostic evaluation is often frustrating for both physicians and patients because a precise anatomical explanation is elusive. Some experts [see, Praemer, A., Furnes, S., Rice, D. P., Musculoskeletal conditions in the United States. 1992: p. 1-99.] suggest it is generally more useful for the physician to address 3 questions: Is a systemic disease causing the pain? Is there social or psychological distress that may amplify or prolong the pain? Is there neurological compromise that may require surgical evaluation? These questions can be addressed through medical history and physical examination., which is within the skill of the typical practitioner.

Point prevalence estimates showed 6.4% of the population suffers from chronic low back pain. Han et al., 2000. Chronic low back pain is a leading reason for physician visits and work disability and costs the U.S. over $50 billion annually in health care costs, disability claims, and lost productivity. Frymoyer, J. W. and W. L. Cats-Baril, An overview of the incidences and costs of low back pain. Orthop Clin North Am, 1991. 22(2): p. 263-71.

Risk factors for chronic low back pain include those within the individual, occupational, and psychosocial domains. See Manek, 2005. Individual risk factors include smoking, obesity, and age. Although the prevalence of chronic low back pain increases with age, the dose-response relation between age and low back pain is not linear, suggesting multiple factors are involved. Women, but not men, who are overweight or with large hip-to-waist ratios have an increased likelihood of developing chronic low back pain. Suzuki et al., 2004.

Sleep disturbances and complaints of poor sleep quality are very common among patients with pain-related conditions. Additionally, sleep improvement is often used as an indicator of pain relief. In chronic low back pain, subjective measures indicate the presence and stability of sleep disturbance; although, objective assessments using polysomnography revealed only subtle differences in sleep quality. Harman, K., et al., Sleep in depressed and nondepressed participants with chronic low back pain: electroencephalographic and behavior findings. Sleep, 2002. 25(7): p. 775-83. Hence, agents that improve sleep could have a beneficial effect in chronic low back pain patients. On the other hand, sedative agents can have deleterious effects on patients during waking hours, interfering with normal activities as well as the operation of heavy equipment, including automobiles. Thus therapeutics that promote sleep but induce daytime sleepiness are considered unsuitable for long term care in many circumstances.

As discussed previously in this document, neural pathways originating from the brainstem suppress sensory transmission and consequently produce analgesia. Suzuki et al., 2004. These descending inhibitory pathways typically utilize 5-HT and NE as neurotransmitters, and this may partially explain why drugs that enhance extracellular levels of 5-HT and NE, such as the tricyclic antidepressants have been found clinically to exhibit analgesic properties in chronic pain conditions.

Central sensitization is a CNS condition that typically occurs following peripheral nerve injury, and consequently neurons in the spinal cord become hyperexcitable and much more responsive to neuronal inputs from the periphery. Suzuki et al., 2004. These inputs are usually too weak to cause excitation under normal circumstances, but in sensitized states, non-noxious stimuli can lead to widespread pain extending beyond the site of damage/stimulation. In chronic low back pain, it has been hypothesized that a process somewhat similar to central sensitization may be responsible for the heightened and long-term pain that occurs in the absence of sustained tissue injury. Arendt-Nielsen, L. and T. Graven-Nielsen, Central sensitization in fibromyalgia and other musculoskeletal disorders. Curr Pain Headache Rep, 2003. 7(5): p. 355-61.

Ion channels also play an important role in mediating chronic pain states. Activation or increased expression of Na⁺ and Ca⁺⁺ channels enhances membrane excitability directly leading to increased neuronal signaling. Nerve injury, which can produce chronic pain, enhances the expression of Na⁺ channels. Priestly et al., 2004. Blockade of Na⁺ channels with lidocaine reduces pain associated with nerve injury both in animal models and in humans. Similarly, Ca⁺⁺ channel subunit expression is also increased following nerve injury and the Ca⁺⁺ channel blocker ziconotide reduces pain in animals and humans. McGivem, J. G. and S. I. McDonough, Voltage-gated calcium channels as targets for the treatment of chronic pain. Curr Drug Targets CNS Neurol Disord, 2004. 3(6): p. 457-78.

Chronic lower back pain has proven to be a difficult therapeutic target, and a number of different pharmacologic approaches have been tried. Tricyclic compounds have demonstrated some degree of efficacy, presumably based on their ability to increase NE and 5-HT. Antiepileptic drugs, including zonisamide, have also been shown to have a beneficial effect on chronic pain states, presumably through their membrane stabilizing effects. These two mechanisms are independent, and some embodiments of the invention provide a combination of a drug that stimulates NE and 5-HT (such as mirtazapine) with a drug that reduces neuronal excitability and ion channel activity (such as zonisamide). In some embodiments of the invention such a combination is synergistic.

Specifically, embodiments of the invention provide that mirtazapine, which has the ability to elevate 5HT and NE, is combined with zonisamide, resulting in a multiple pathway approach to pain reduction. In some embodiments this treatment regime is beneficial, especially where treatment of chronic low back pain is particularly intractable. Both drugs, when administered alone, have analgesic properties; and it is an object of the invention that the combination of the two produce effective analgesia in CLBP by targeting multiple key receptors and pathways involved in chronic pain processing. In addition, zonisamide has the advantage of being a mild stimulant. Thus, it is a further object of the invention to provide analgesia while reducing or avoiding the side-effects associated with use of mirtazapine alone, such as sedation, excessive daytime sleepiness and weight gain.

Mirtazapine's ability to elevate 5HT and NE suggests its utility in treating chronic pain because drugs with similar effects (tricyclic antidepressants, NSRIs) produce beneficial responses in alleviating chronic pain. Although there are no published trials in chronic low back pain, an open study suggested that mirtazapine has some activity in fibromyalgia pain [Samborski, W., M. Lezanska-Szpera, and J. K. Rybakowski, Open trial of mirtazapine in patients with fibromyalgia. Pharmacopsychiatry, 2004. 37(4): p. 168-70] and a controlled trial suggested efficacy in chronic tension type headache. Bendtsen, L. and R. Jensen, Mirtazapine is effective in the prophylactic treatment of chronic tension-type headache. Neurology, 2004. 62(10): p. 1706-11. An open trial in cancer pain, however, found that, although significant improvements were obtained in depression levels, pain intensity was not statistically improved, although a trend for improvement was apparent. Theobald, D. E., et al., An open-label, crossover trial of mirtazapine (15 and 30 mg) in cancer patients with pain and other distressing symptoms. J Pain Symptom Manage, 2002. 23(5): p. 442-7. In addition to mirtazapine's ability to elevate both 5HT and NE, its 5HT3 blocking activity could also useful for reducing pain, based on clinical studies with the 5HT3 antagonist tropisetron in low back pain.

As mentioned above, poor sleep quality is a common complaint of patients with chronic low back pain. Mirtazapine has sleep-promoting properties and is often prescribed to depressed patients with insomnia. Clinical studies have found that mirtazapine improves objective and subjective sleep measures, in both depressed patients and in normal subjects, including sleep onset, total sleep time, and sleep efficiency. Aslan, S., E. Isik, and B. Cosar, The effects of mirtazapine on sleep: a placebo controlled, double-blind study in young healthy volunteers. Sleep, 2002. 25(6): p. 677-9. Winokur, A., et al., Acute effects of mirtazapine on sleep continuity and sleep architecture in depressed patients: a pilot study. Biol Psychiatry, 2000. 48(1): p. 75-8. The analgesic and somnolence-promoting effects of mirtazapine may provide additive benefits in chronic low back pain.

Although no trials of zonisamide in chronic low back pain have been published to date, zonisamide has been shown to be effective in cases of neuropathic pain. Guay, D. R., Oxcarbazepine, topiramate, zonisamide, and levetiracetam: potential use in neuropathic pain. Am J Geriatr Pharmacother, 2003. 1(1): p. 18-37. Zonisamide (100-200 mg/d) was reported to improve subjective ratings of pain by at least 50% in 59% of the patients examined with neuropathic pain. Hasegawa, H., Utilization of zonisamide in patients with chronic pain or epilepsy refractory to other treatments: a retrospective, open label, uncontrolled study in a VA hospital. Curr Med Res Opin, 2004. 20(5): p. 577-80. As mentioned above, both Na⁺ and Ca⁺⁺ channels are involved in neural processing of chronic pain states, and the ability of zonisamide to block Na⁺ and Ca⁺⁺ channels likely explains its pain-reducing qualities.

Reductions in body weight could also be of benefit, since obesity can increase the risk of chronic low back pain, particularly in women. In short term clinical trials of zonisamide in epileptic patients also receiving other anti-convulsants, weight loss was an adverse effect. Oommen, K. J. and S. Mathews, Zonisamide: a new antiepileptic drug. Clin Neuropharmacol, 1999. 22(4): p. 192-200. In a randomized, controlled trial in obese patients, zonisamide reduced body weight by 6% (versus 1% loss in placebo-treated subjects) following 16 weeks of therapy. Gadde, K. M., et al., Zonisamide for weight loss in obese adults: a randomized controlled trial. Jama, 2003. 289(14): p. 1820-5. In the 32 week extension phase of this study, obese patients receiving zonisamide exhibited 9.4% loss of body weight versus 1.8% loss for patients on placebo. Zonisamide can also reduce weight gain associated with the use of psychiatric drugs. For example, zonisamide co-therapy reduced the increase in body weight produced by the anti-psychotic drug risperidone. Krishnan, K. R. 2004, personal communication. In summary, zonisamide has weight-loss promoting properties and has utility in treating obese patients and cases of anti-psychotic-induced weight gain.

Conversely, mirtazapine's use has been associated with increases in appetite and body weight. In controlled studies, appetite increase was reported in 17% of patients treated with mirtazapine, compared to 2% for placebo, and 6% for amitriptyline. In these same trials, weight gain of greater than or equal to 7% of body weight was reported in 7.5% of patients treated with mirtazapine, compared to 0% for placebo and 5.9% for amitriptyline. Results from a long-term trial with mirtazapine in depressed patients suggests that the greatest weight gain occurs during the initial 12 weeks of treatment with only slight weight gain occurring during the 40 week extension phase. Krishnan, K. R. 2004, personal communication. While mirtazapine may increase appetite and body weight when administered alone zonisamide in combination with mirtazapine may counteract this potential adverse effect.

In some embodiments, then mirtazapine and zonisamide in combination provide superior analgesia in patients with chronic low back pain. Moreover, in some embodiments mirtazapine and zonisamide in combination have limited side effects compared to other drugs currently in use, or at least reduced side effects when compared to either drug taken separately. Multiple receptors and neural pathways are involved in pain processing, and the combination of mirtazapine and zonisamide affects multiple targets, including 5HT, NE, 5HT3 receptors, Na⁺ channels, and Ca⁺⁺ channels. In addition, mirtazapine has important sleep-promoting properties, and, while it alone may increase appetite and body weight, in some embodiments the addition of zonisamide tends to offset this effect.

B. Sleep-Related Breathing Disorders

Over the past several years much effort has been devoted to the study of a discrete group of breathing disorders that occur primarily during sleep with consequences that may persist throughout the waking hours in the form of sleepiness, thereby manifesting itself into substantial economic loss (e.g., thousands of lost man-hours) or employment safety factors (e.g., employee non-attentiveness during operation of heavy-machinery). Sleep-related breathing disorders are characterized by repetitive reduction in breathing (hypopnea), snoring, periodic cessation of breathing (apnea), or a continuous or sustained reduction in ventilation.

In general sleep apnea is defined as an intermittent cessation of airflow at the nose and mouth during sleep. Sleep apnea has been linked to serious medical conditions such as heart disease, hypertension, stroke, obesity, and decreased pulmonary function. In severe cases sleep apnea may even cause death. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas (OSAS), airflow ceases despite continuing respiratory drive because of occlusion of the oropharyngeal airway. Mixed apneas, which consist of a central apnea followed by an obstructive component, are a variant of obstructive sleep apnea. The most common type of apnea is obstructive sleep apnea.

Hypopneas are episodes of shallow breathing during which airflow is decreased by at least 50%. Like apnea, hypopnea is subdivided as being obstructive, central, or mixed. Obstructive hypopneas are episodes of partial upper airway obstruction. In central hypopnea, breathing effort and airflow are both decreased. Mixed hypopneas have both central and obstructive components. Individuals with OSA syndrome have pathologic degrees of obstructive apnea, obstructive hypopnea, or both.

The term Upper Airway Resistance Syndrome (UARS) is used to describe chronic daytime sleepiness in the absence of actual apneas or hypopneas, but often associated with snoring, and with brief, frequent arousals with an only slightly abnormal breathing pattern. Patients with the clinical features of apnea, hypopnea and nocturnal oxygen desaturation during polysomnography (PSG).

Patients with UARS lack the typical findings of apnea on PSG, and therefore, are often not diagnosed. The arousals and sleep fragmentation are related to an increased effort to breathe which can be diagnosed by measurement of pressure changes in the esophagus.

The term “snoring” generally refers to a rough or hoarse sound that arises from a person's mouth during sleep. Snoring is believed to be generally caused by the narrowing of the pharyngeal airway such that turbulent airflow during relaxed breathing vibrates the soft parts of the pharyngeal passage, such as the soft palate, the posterior faucial pillars of the tonsils and the uvula. A restricted pharyngeal passageway can occur anatomically. For example, in children, this often is caused by obstruction due to enlarged tonsils or adenoids. In adults, it is not unusual for the narrowing to be caused by obesity. Further anatomical narrowing can be simple a matter of heredity, with some persons being predisposed towards a smaller pharyngeal cross-section. A reduced pharyngeal passageway may also be caused by a lack of muscle tone.

Snoring can indicate a more serious condition and, due to exhaustion resulting from lack of sleep, can cause other problems. For example, an association between snoring and coronary artery disease and hypertension has been found, and cardiac arrhythmia has been reported during sleep apnea attacks. As stated above, people with sleep apnea often snore, however, sleep apnea can also be present without snoring. Not only is the risk of cessation of breathing dangerous, lack of oxygen due to an obstructed pharyngeal passageway deprives the body of sufficient oxygen so that oxygen desaturation arises. Lack of oxygen may cause the brain to rouse the sleeper just enough to take a breath without fully awaking. This may occur hundreds of times a night, with the result that the snorer fails to get sufficient sleep. Moreover, being aroused from deep REM sleep on a repetitive basis may increase heart rate and blood pressure. Thus, snoring may increase the risk of heart attack and stroke (Leineweber et al. Sleep 27(7): 1344-1349 (2004)).

C. Depression

Depression refers to an abnormal mood or a collection of symptoms that constitute a psychiatric disorder. Symptoms of depression include disturbances in mood and affect (depressed mood, diminished interest and pleasure in activities), bodily function (weight and appetite changes, psychomotor disturbances, sleep disturbances, fatigue and loss of energy), and cognitive processes (feelings of worthlessness and guilt, concentration difficulties, indecisiveness, thoughts of death or suicide and possibly delusions/hallucinations). These symptoms vary in intensity, duration and frequency and permit classification of depression into different classes. Other symptoms of major depressive episodes include crying spells, self-pity, hopelessness, irritability, brooding, diminished self-esteem, decreased libido, nihilism, social withdrawal, memory impairment, feelings of inadequacy and pessimism. These symptoms are summarized in the American Psychiatric Association's Diagnostic and Statistical Manual of Mental Disorders, 4th Edition, Text Revision (DSM-IV-TR; 1994).

Atypical depression is one type of depressive disorder included in DSM-IV-TR at page 420 about which there has been substantial clinical and research interest. Although at the present time it is not clear how common this diagnosis is in chronic pain patients, there are certainly pain patients expressing the characteristics of atypical depression.

There are at least two broad types of atypical depression that differ from classically defined depression (Davidson et al. Arch. Gen. Psychiatry, 39, 527-34 (1982); Paykel et al. Psychol. Med., 13,:131-9 (1983); Paykel et al, Arch. Gen. Psychiatry, 39:1041-9 (1982)). One is composed of those depressions accompanied by severe anxiety, and also by phobic symptoms, tension, and pain. The other type of atypical depression is characterized by reversed vegetative symptoms, e.g., increased (rather than decreased) appetite, weight, and sleep.

D. Schizophrenia

Schizophrenia is a devastating brain disorder that affects approximately 2.2 million American adults, or 1.1 percent of the population age 18 and older. Schizophrenia interferes with a person's ability to think clearly, to distinguish reality from fantasy, to manage emotions, make decisions, and relate to others. The first signs of schizophrenia typically emerge in the teenage years or early twenties. Most people with schizophrenia suffer chronically or episodically throughout their lives, and are often stigmatized by lack of public understanding about the disease.

The symptoms of schizophrenia are generally divided into three categories, including positive, disorganized and negative symptoms. Positive Symptoms, or “psychotic” symptoms, include delusions and hallucinations because the patient has lost touch with reality in certain important ways. Disorganized Symptoms include confused thinking and speech, and behavior that does not make sense. Negative Symptoms include emotional flatness or lack of expression, an inability to start and follow through with activities, speech that is brief and lacks content, and a lack of pleasure or interest in life.

Schizophrenia is also associated with changes in cognition. These changes affect the ability to remember and to plan for achieving goals. Attention and motivation are also diminished. The cognitive problems of schizophrenia may be important factors in long term outcome.

Schizophrenia also affects mood. Many individuals affected with schizophrenia become depressed, and some individuals also have apparent mood swings and even bipolar-like states. When mood instability is a major feature of the illness, it is called, schizoaffective disorder, meaning that elements of schizophrenia and mood disorders are prominently displayed by the same individual. It is not clear whether schizoaffective disorder is a distinct condition or simply a subtype of schizophrenia.

E. Anxiety Disorders

1. Generalized Anxiety Disorder

Most people experience anxiety at some point in their lives and some nervousness in anticipation of a real situation. However if a person cannot shake unwarranted worries, or if the feelings are jarring to the point of avoiding everyday activities, he or she most likely has an anxiety disorder. Symptoms include chronic, exaggerated worry, tension, and irritability that appear to have no cause or are more intense than the situation warrants. Physical signs, such as restlessness, trouble falling or staying asleep, headaches, trembling, twitching, muscle tension, or sweating, often accompany these psychological symptoms.

2. Panic Disorder

People with panic disorder experience white-knuckled, heart-pounding terror that strikes suddenly and without warning. Since they cannot predict when a panic attack will seize them, many people live in persistent worry that another one could overcome them at any moment. Symptoms include pounding heart, chest pains, lightheadedness or dizziness, nausea, shortness of breath, shaking or trembling, choking, fear of dying, sweating, feelings of unreality, numbness or tingling, hot flashes or chills, and a feeling of going out of control or going crazy.

3. Phobias

Phobias are irrational fears that lead people to altogether avoid specific things or situations that trigger intense anxiety. Phobias occur in several forms, for example, agoraphobia is the fear of being in any situation that might trigger a panic attack and from which escape might be difficult. Social Phobia or Social Anxiety Disorder is the fear of social situations and the interaction with other people, which can automatically bring on feelings of self-consciousness, judgment, evaluation, and criticism. It is the fear and anxiety of being judged and evaluated negatively by other people, leading to feelings of inadequacy, embarrassment, humiliation, and depression. Many of the physical symptoms that accompany panic attacks—such as sweating, racing heart, and trembling—also occur with phobias.

4. Post-Traumatic Stress Disorder

Anyone can develop Post-traumatic Stress Disorder (PTSD) if they have experienced, witnessed, or participated in a traumatic occurrence-especially if the event was life threatening. PTSD can result from terrifying experiences such as rape, kidnapping, natural disasters, war or serious accidents such as airplane crashes. The psychological damage such incidents cause can interfere with a person's ability to hold a job or to develop intimate relationships with others. The symptoms of PTSD can range from constantly reliving the event to a general emotional numbing. Persistent anxiety, exaggerated startle reactions, difficulty concentrating, nightmares, and insomnia are common. People with PTSD typically avoid situations that remind them of the traumatic event, because they provoke intense distress or even panic attacks.

F. Insomnia

Insomnia is chronic and persistent difficulty in either (1) falling asleep (initial insomnia), (2) remaining asleep through the night (middle insomnia), or (3) waking up too early (terminal insomnia). All types of insomnia can lead to daytime drowsiness, poor concentration, and the inability to feel refreshed and rested in the morning.

There are several types of insomnia. Sleep-onset insomnia occurs when people have difficulty falling asleep because they think and worry and cannot let their minds relax. Sleep maintenance insomnia occurs when people fall asleep normally but wake up several hours later and cannot fall asleep again easily. Sometimes they drift in and out of a restless, unsatisfactory sleep. Early morning awakening, another type of insomnia, may be a sign of depression in people of any age.

Sleep-wake schedule disorder may occur in people whose sleep patterns have been disrupted: They fall asleep at inappropriate times and then cannot sleep when they should. These sleep-wake reversals often result from jet lag (especially when traveling from east to west), working irregular night shifts, frequent changes in work hours, or excessive use of alcohol. Sometimes sleep-wake reversals are a side effect of drugs. Sleep-wake reversals are common among people who are hospitalized because they are often awakened during the night. Damage to the brain's built-in biologic clock (caused by encephalitis, stroke, or Alzheimer's disease, for example) can also disrupt sleep patterns.

G. Headaches

1. Tension-Type headaches

Tension type headaches are the most common, affecting upwards of 75% of all headache sufferers. Tension-type headaches are typically a steady ache rather than a throbbing one and affect both sides of the head. Tension-type headaches may also be chronic, occurring frequently or even every day.

2. Migraine Headaches

Migraine headaches are less common than tension-type headaches. Nevertheless, migraines afflict 25 to 30 million people in the United States alone. Migraines are felt on one side of the head by about 60% of migraine sufferers, and the pain is typically throbbing in nature. Migraines are often accompanied by nausea and sensitivity to light and sound. A group of telltale neurologic symptoms known as an aura, sometimes occurs before the head pain begins. Typically, an aura involves a disturbance in vision that may consist of brightly colored or blinking lights in a pattern that moves across the field of vision. Usually, migraine attacks are occasional, or sometimes as often as once or twice a week, but not daily.

3. Cluster Headaches

Cluster headaches are relatively rare, affecting about 1% of the population, and are distinct from migraine and tension-type headaches. Cluster headaches come in groups or clusters lasting weeks or month. The pain is extremely severe, but the attack is brief, lasting no more than an hour or two. The pain centers around one eye, and this eye may be inflamed and watery. There may also be nasal congestion on the affected side of the face. These headaches may strike in the middle of the night, and often occur at about the same time each day during the course of a cluster.

H. Hot Flashes

Approximately 85% of women will experience hot flashes to some degree. Also known as hot flushes, these symptoms appear because of changing hormone levels around the time of menopause. For some women, hot flashes are nothing more than a mild and fleeting sensation of warmth, but for others hot flashes cause frequent, intense discomfort. Typically, a hot flash starts with increased blood flow to the extremities, increased heart rate and anxiety. A noticeable flush appears on the face and chest, and the sensation of heat may be pronounced. The profuse sweating that often accompanies a hot flash can be a source of stress and social embarrassment, and may interfere with restful sleep.

The precise mechanism responsible for hot flashes is not known for certain, but hormone fluctuations are thought to be a significant factor. Underweight women tend to experience more frequent hot flashes, possibly because fat plays a supportive role in hormone production. In addition, hot flashes affect smokers earlier in life than nonsmokers.

I. Functional Somatic Syndromes

1. Chronic Fatigue Syndrome

Chronic fatigue syndrome (CFS) is a debilitating disorder characterized by profound tiredness or fatigue. Patients with CFS may become exhausted with only light physical exertion, and must often function at a level of activity substantially lower than their capacity before the onset of illness. In addition to the key defining characteristic of fatigue, CFS patients generally report various nonspecific symptoms, including weakness, muscle aches and pains, excessive sleep, malaise, fever, sore throat, tender lymph nodes, impaired memory and/or mental concentration, insomnia, and depression. Like patients with fibromyalgia, patients with CFS suffer from disordered sleep, localized tenderness, and complaints of diffuse pain and fatigue.

There are two widely used criteria for diagnosing CFS. The criteria established by the U.S. Centers for Disease Control and Prevention include medically unexplained fatigue of at least six months duration that is of new onset, not a result of ongoing exertion and not substantially alleviated by rest, and a substantial reduction in previous levels of activity. In addition, the diagnosis involves the determination of the presence of four or more of the following symptoms—subjective memory impairment, tender lymph nodes, muscle pain, joint pain, headache, unrefreshing sleep, and postexertional malaise (>24 hours) (Reid et al., 2000, British Medical Journal 320: 292-296). The diagnostic criteria from Oxford includes severe, disabling fatigue of at least six months duration that affects both physical and mental functioning and the fatigue being present for more than 50% of the time. In addition, the diagnosis involves the determination of the presence of other symptoms, particularly myalgia and sleep and mood disturbance (Reid et al., 2000, British Medical Journal 320: 292-296).

2. Fibromyalgia Syndrome

Fibromyalgia syndrome (FMS) is the most frequent cause of chronic, widespread pain, estimated to affect 2-4% of the population. FMS is characterized by a generalized heightened perception of sensory stimuli. Patients with FMS display abnormalities in pain perception in the form of both allodynia (pain with innocuous stimulation) and hyperalgesia (increased sensitivity to painful stimuli). The syndrome, as defined by the American College of Rheumatology's criteria, involves the presence of pain for over 3 months duration in all four quadrants of the body, as well as along the spine. In addition, pain is elicited at 11 out of 18 “tender points” upon palpation. Other associated symptoms include fatigue, nonrestorative sleep, and memory difficulties.

Owing to their common symptomology, FMS and CFS are thought to be related. However, they manifest different major symptoms. Whereas pain is the major symptom reported by patients with FMS, fatigue is the major symptom reported by patients with CFS. Given their relatedness, these two indications have been treated with the same medications.

3. Irritable Bowel Syndrome

Irritable bowel syndrome (IBS) is a gastrointestinal disorder characterized by continuous or recurrent abdominal pain or discomfort that is relieved with defecation and is associated with a change in the consistency or frequency of stool. IBS has elements of an intestinal motility disorder, a visceral sensation disorder, and a central nervous disorder. While the symptoms of IBS have a physiological basis, no physiological mechanism unique to IBS has been identified. Epidemiological surveys have estimated the prevalence of IBS ranges from 10-22% of the population with a higher frequency of occurrence in women. Psychological factors, either stress or overt psychological disease, modulate and exacerbate the physiological mechanisms that operate in IBS (Drossman, D. A. et al., Gastroenterology 1988 95:701-708).

Due to a lack of readily identifiable structural or biochemical abnormalities in this syndrome, the medical community has developed a consensus definition and criteria, known as the Rome criteria, to aid in diagnosis of IBS. According to the Rome criteria, IBS is indicated by abdominal pain or discomfort which is (1) relieved by defection and/or (2) associated with a change in frequency or consistency of stools, plus two or more of the following: altered stool frequency, altered stool form, altered stool passage, passage of mucus, and bloating or feeling of abdominal distention (Dalton, C. and Drossman, D. A., Am Fam Physician 1997 55(3):875-880). Thus, a hallmark of IBS is abdominal pain that is relieved by defecation, and which is associated with a change in the consistency or frequency of stools. IBS may be diarrhea-predominant, constipation-predominant, or an alternating combination of both.

Non-gastrointestinal symptoms are common and increase in number as the severity of IBS increases. Chronic fatigue, headache, urological symptoms and other multi-system complaints occur including fibromyalgia.

J. Lower Back Pain (Other than Chronic Lower Back Pain)

Aside from chronic lower back pain, which is a functional somatic disorder, other common causes of lower back pain include lumbar strain, nerve irritation, lumbar radiculopathy, bony encroachment, and conditions of the bone and joints.

Lumbar Strain—A lumbar strain is a stretching injury to the ligaments, tendons, and/or muscles of the lower back. The stretching incident results in microscopic tears of varying degrees in these tissues. Lumbar strain is considered one of the most common causes of low back pain. The injury can occur because of overuse, improper use, or trauma. Soft tissue injury is commonly classified as “acute” if it has been present for days to weeks. If the strain lasts longer than 3 months, it is referred to as “chronic.” Lumbar strain most often occurs in persons in their forties, but can happen at any age. The condition is characterized by localized discomfort in the lower back area with onset after an event that mechanically stressed the lumbar tissues. The severity of the injury ranges from mild to severe, depending on the degree of strain and resulting spasm of the muscles of the lower back.

Nerve Irritation—The nerves of the lumbar spine can be irritated by mechanical impingement or disease any where along their paths—from their roots at the spinal cord to the skin surface. These conditions include lumbar disc disease (radiculopathy), bony encroachment, and inflammation of the nerves caused by a viral infection (shingles).

Lumbar Radiculopathy—Lumbar radiculopathy refers to nerve irritation which is caused by damage to the discs between the vertebrae. Damage to the disc occurs because of degeneration (“wear and tear”) of the outer ring of the disc, traumatic injury, or both. As a result, the central softer portion of the disc can rupture (herniate) through the outer ring of the disc and abut the spinal cord or its nerves as they exit the bony spinal column. This rupture is what causes the commonly recognized “sciatica” pain that shoots down the leg. Sciatica can be preceded by a history of localized low back aching or it can follow a “popping” sensation and be accompanied by numbness and tingling. The pain commonly increases with movements at the waist and can increase with coughing or sneezing. In more severe instances, sciatica can be accompanied by incontinence of the bladder and/or bowels.

Bony Encroachment—Any condition that results in movement or growth of the vertebrae of the lumbar spine can limit the space (encroachment) for the adjacent spinal cord and nerves. Causes of bony encroachment of the spinal nerves include foraminal narrowing (narrowing of the portal through which the spinal nerve passes from the spinal column, out of the spinal canal to the body), spondylolisthesis (slippage of one vertebra relative to another), and spinal stenosis (compression of the nerve roots or spinal cord by bony spurs or other soft tissues in the spinal canal). Spinal nerve compression in these conditions can lead to sciatica pain which radiates down the lower extremities. Spinal stenosis can cause lower extremity pains which worsen with walking and are relieved by resting (mimicking poor circulation).

Bone & Joint Conditions—Bone and joint conditions that lead to low back pain include those existing from birth (congenital), those that result from wear and tear (degenerative) or injury, and those that are from inflammation of the joints (arthritis).

Congenital causes (existing from birth) of low back pain include scoliosis and spina bifida. Scoliosis is a sideways (lateral) curvature of the spine which can be caused when one lower extremity is shorter than the other (functional scoliosis) or because of an abnormal design of the spine (structural scoliosis). Spina bifida is a birth defect in the bony vertebral arch over the spinal canal, often with absence of the spinous process. This birth defect most commonly affects the lowest lumbar vertebra and the top of the sacrum.

As we age, the water and protein content of the body's cartilage changes. This change results in weaker, thinner, and more fragile cartilage. Because both the discs and the joints that stack the vertebrae (facet joints) are partly composed of cartilage, these areas are subject to wear and tear over time (degenerative changes). Degeneration of the disc is called spondylosis. Spondylosis can be noted on x-rays of the spine as a narrowing of the normal “disc space” between the vertebrae. It is the deterioration of the disc tissue that predisposes the disc to herniation and localized lumbar pain (“lumbago”) in older patients. Degenerative arthritis (osteoarthritis) of the facet joints is also a cause of localized lumbar pain that can be detected with plain x-ray testing. These causes of degenerative back pain are usually treated conservatively with intermittent heat, rest, rehabilitative exercises, and medications to relieve pain, muscle spasm, and inflammation.

Fractures (breakage of bone) of the lumbar spine and sacrum bone most commonly affect elderly persons with osteoporosis, especially those who have taken long-term cortisone medication. For these individuals, occasionally even minimal stresses on the spine (such as bending to tie shoes) can lead to bone fracture. In this setting, the vertebra can collapse (vertebral compression fracture). The fracture causes an immediate onset of severe localized pain that can radiate around the waist in a band-like fashion and is made intensely worse with body motions.

The spondyloarthropathies are inflammatory types of arthritis that can affect the lower back and sacroiliac joints. Examples of spondyloarthropathies include Reiter's disease, ankylosing spondylitis, psoriatic arthritis, and the arthritis of inflammatory bowel disease. Each of these diseases can lead to pain and stiffness in the lower back which is typically worse in the morning. These conditions usually begin in the second and third decades of life.

K. Neuropathic Pain

Neuropathic pain may result from a wide spectrum of insults to the peripheral or central nervous system. This may include nutritional deficiencies, systemic diseases, chemotherapy, cerebrovascular accident, surgery or trauma. The hallmark of neuropathic pain is abnormal neural activity in peripheral nerve(s) or the central nervous system. This is often accompanied by disordered sensory processing both in the peripheral or central nervous system. Even in injuries which are primarily peripheral in their location, the central nervous system often becomes involved. The pain frequently has burning, lancinating, or electric shock qualities. Persistent allodynia, pain resulting from a non-painful stimulus such as a light touch, is also a common characteristic of neuropathic pain. The pain may persist for months or years beyond the apparent healing of any damaged tissues.

III. Side Effects Associated with 5HT2/5HT3 Antagonist/Alpha-2 Antagonist Use

A. Excessive Daytime Sleepiness and Weight Gain

Mirtazapine use in the treatment of disorders such as depression, schizophrenia, anxiety disorders, sleep-related breathing disorders, insomnia, migraine headache, chronic tension-type headache, hot flashes, and functional somatic syndromes can cause excessive daytime sleepiness and weight gain in a patient by its sedating effects. The drug is usually given at night, however, because of its long half-life, it can cause sleepiness or fatigue during the day. This often contributes to weight gain by reducing an individual's daily physical activity level.

The symptoms of excessive daytime sleepiness include an overwhelming desire to sleep during what should be waking hours, the need for frequent naps, the inability to concentrate, falling asleep during meetings, class, at work or driving. People find that excessive daytime sleepiness can interfere with their ability to be productive and maintain healthy social relationships. They sometimes feel low self-esteem, frustration, and anger at oneself caused by the disorder and are sometimes misunderstood as being lazy or unintelligent.

IV. Methods of Use

A. Administration Protocol

The 5HT2/5HT3 antagonist/alpha-2 antagonist compositions are administered in an effective dosage to alleviate the symptoms of a disorder and the dopamine-releasing compositions, anticonvulsants, or dopamine/norepinephrine reuptake inhibitors are administered in combination with the 5HT2/5HT3 antagonist/alpha-2 antagonist in an effective dosage to reduce the side effects associated with the 5HT2/5HT3 antagonist/alpha-2 antagonist. The compositions will typically be administered orally. In one embodiment, the 5HT2/5HT3 antagonist/alpha-2 antagonist and dopamine releasing compound, anticonvulsant, or dopamine/norepinephrine reuptake inhibitor are administered simultaneously. In another embodiment, the dopamine-releasing compound, anticonvulsant, or dopamine/norepinephrine reuptake inhibitor is not administered until at least 6 hours after the 5HT2/5HT3 antagonist/alpha-2 antagonist. The compositions can be administered as immediate release, sustained release, intermittent release, and/or delayed release formulations. The composition can be administered in a single dose, an escalating dose, or administered at an elevated dosage which is then decreased to a lower dosage after a particular circulating blood concentration of the compound has been achieved.

An intermittent administration protocol may be used where chronic administration is not desirable. The compound or formulation is administered in time blocks of several days with a defined minimum washout time between blocks. Intermittent administration occurs over a period of several weeks to months to achieve a significant improvement in the symptoms of the disorders.

One of skill in the art would be able to choose administration protocols and determine appropriate dosing regimes to treat symptoms of sleep-related breathing disorders based on bioavailability and half-life of the compound to be administered. For many of the disclosed compounds, appropriate dosage ranges have been established to maximize circulating concentrations of the compound and minimize side-effects.

The 5HT2/5HT3 antagonist/alpha-2 antagonist can be administered for a specific duration to improve symptoms of a particular disorder. A suitable endpoint can be where one symptom of the disorder is treated by administration of the compound and the treatment considered effective. In other situations, the treatment can be considered effective when more than one symptom is treated. The dopamine-releasing compound, anticonvulsant, or dopamine/norepinephrine reuptake inhibitor can be administered in combination with the 5HT2/5HT3 antagonist/alpha-2 antagonist for the duration of use of the 5HT2/5HT3 antagonist/alpha-2 antagonist or even after treatment has been discontinued. A suitable endpoint can be where one side effect of the 5HT2/5HT3 antagonist/alpha-2 antagonist is treated by administration of the dopamine-releasing compound or anticonvulsant and the treatment considered effective. In other situations, the treatment can be considered effective when more than one side effect is treated.

B. Effective Dosage Ranges

Appropriate dosages can be determined by one of skill in the art based on using routine experimentation and standard techniques utilizing dosages currently approved. Compounds in the disclosed drug classes are known in the art and can be initially administered at similar doses and titrated appropriately to treat symptoms of the disorders and side effects in a given patient. Intra-patient variability is known in the art depending on the severity of symptoms and dosages are commonly adjusted to exact a particular therapeutic effect in a particular patient.

Therapeutically effective amounts for use in humans can also be determined from animal models. For example, a dose for humans can be formulated to achieve a circulating concentration that has been found to be effective in animals. Effective amounts for use in humans can also be determined from human data for the compounds used to treat other disorders, for example, neurological disorders. The amount administered can be the same amount administered to treat other neurological disorders or can be an amount higher or lower than the amount administered to treat other neurological disorders.

The optimal concentration of the drug in each pharmaceutical formulation varies according to the formulation itself. Typically, the pharmaceutical formulation contains the drug at a concentration of about 0.1 to 90% by weight (such as about 1-20% or 1-10%). Appropriate dosages of the drug can readily be determined by those of ordinary skill in the art of medicine by assessing amelioration of the disorder or side effect in the patient, and increasing the dosage and/or frequency of treatment as desired. The optimal amount of the drug may depend upon the mode of administration, the age and the body weight of the patient, and the condition of the patient. In some embodiments, the drugs are administered at a dosage of 0.001 to 100 mg/kg of body weight of the patient; e.g., the drug is administered at a dosage of 0.01 mg to 10 mg/kg or 0.1 to 1.0 mg/kg. Preferred daily doses of the 5HT2/5HT3 antagonist/alpha-2 antagonist (mirtazapine) are approximately 7.5 to 200 mg/day, and preferably 15 to 45 mg/day. Preferred daily doses of setiptiline are generally from about 1 to about 50, especially about 5 to about 20 mg/day. Preferred daily doses of the anticonvulsant (zonisamide) are approximately 10 to 600 mg/day, and preferably 50 to 400 mg/day. Preferred daily doses of the dopamine-releasing compound (amantadine) and the dopamine/norepinephrine reuptake inhibitor (bupropion) are approximately 50 to 400 mg/day.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

The invention may be further appreciated upon consideration of the following illustrative, non-limiting examples.

### EXAMPLE 1

### The Efficacy of Centrally-Acting Analgesics for the Treatment of the Pain Associated with the Chronic Low Back Pain Syndrome

In order to assess the efficacy of a combination of mirtazapine and zonisamide in the treatment of the pain associated with chronic low back pain syndrome, a four arm, randomized, double blind, placebo-controlled study of up to 160 patients is conducted. The efficacy of the combination of mirtazapine and zonisamide is compared with that of diphenhydramine (as an active placebo) and amitriptyline (as an active control). Patients are randomized into one of four equally sized study arms and receive either placebo, amitriptyline, a first, lower dose combination of mirtazapine (15 mg) and zonisamide (100 mg), or a second, higher dose combination of mirtazapine (30 mg) and zonisamide (200 mg). All medications are administered once per day in an over-encapsulated format that ensures blinding of study participants, staff and investigators. The study includes a dose titration phase prior to reaching the stable dosing phase of the trial as well as a drug tapering phase after the end of the stable dosing phase.

All patients are scheduled to receive a total of 14 weeks of therapy, including up to 4 weeks of upward dose titration, 8 weeks of stable dose therapy, and 2 weeks of downward dose titration. Patients who do not tolerate the full upward titration of dosage may stop at the highest tolerated dosage level, and continue the trial at that dosage, or at a lower dosage.

Patients are required to complete paper self-assessments, electronic diary assessments, activity monitoring, salivary cortisol, heart rate variability, psychophysical assessments of pain and functional imaging.

Mirtazapine is administered as over-encapsulated 15 mg tablets. Zonisamide is administered as over-encapsulated 100 mg capsules. Amitriptyline is administered as over-encapsulated 10 and 25 mg tablets. Diphenhydramine is administered as an over-encapsulated capsule containing 25 mg of active placebo. All capsules are identically over-encapsulated in order to maintain blinding of patients, staff and investigators. All capsules are identical in appearance by means of over-encapsulation utilizing size #0 hard, Swedish orange, gelatin capsules.

Clinical Endpoints

Efficacy of the combination of the combination of mirtazapine and zonisamide is assessed using the following methods:

Self-reporting questionnaires

Electronic patient experience diary (PED)

Activity monitoring

Psychophysical assessment of pain

Functional imaging (e.g. functional MRI).

Self-Reporting Questionnaires

Symptom Profile. Chronic low back pain (CLBP) and symptoms associated with related chronic multisymptom illnesses (pain, fatigue, sleep disturbance, and memory complaints) are assessed using validated assessment tools for these conditions. All of the following self-report instruments are administered at the baseline study visit (week BL2/Tx0) and at Weeks Tx6 and Tx12 of participation. Self report forms are described below.

CLBP symptoms are assessed using the following 3 measures:

Roland-Morris: The Roland-Morris Questionnaire (RMQ) is a self-administered disability measure in which greater levels of disability are reflected by higher numbers on a 24-point scale. It focuses on activity intolerances related to one's low back problem and was developed from the Sickness Impact Profile (SIP), a widely used disability questionnaire. [Roland M, M. R., A study of the natural history of back pain. Part I. Development of a reliable and sensitive measure of disability in low-back pain, Spine, 1983, 8(2)(March), 141-4.] The RMQ yields reliable measurements, which are valid for inferring the level of disability, and is sensitive to change over time for groups of patients with low back pain. It is well suited to administration by telephone and therefore is invaluable in research when securing follow-up information efficiently. [Deyo R. A., et al., Outcome measures for low back pain research. A proposal for standardized use, Spine, 1998, 23(18)(September 15), 2003-13.]

Oswestry Low Back Disability Questionnaire: The OSW was originally described in 1980. [Fairbank J C, C. J., Davies J B, O'Brien J P, The Oswestry low back pain disability questionnaire. Physiotherapy, 1980, 66(8)(August), 271-3.] The questionnaire consists of 10 items addressing different aspects of function. Each item is scored from 0 to 5, with higher values representing greater disability. The total score is multiplied by 2 and expressed as a percentage.

Multidimensional Pain Inventory: The Multidimensional Pain Inventory (MPI), Version II, is a 61 item inventory which is divided into 3 parts with several subscales in each part. [Kerns R D, T. D., Rudy T E, The West Haven-Yale Multidimensional Pain Inventory (WHYMPI), Pain, 1985, 23(4)(December), 345-56.] Part I examines 5 dimensions of the pain experience (perceived interference of pain in various areas of patients' functioning, support and concern of significant others, pain severity, self-control, and negative affect). Part II evaluates the responses of significant others to communication of pain, and includes three subscales (perceived frequency of punishing, solicitous, and distracting responses). Part III assesses participation in 4 categories of daily activities (household chores, outdoor work, activities away from home and social activities).

Pain, either evoked or clinical (using the electronic pain diaries) is assessed using several scales. The measures include the Box scale, which has been used with evoked pain testing and fMRI studies, as well as clinical studies involving electronic pain diaries with FM patients. Pain is also assessed via the Patient Experience Diary (PED), a PDA based, real-time symptom collection tool. The PED is described in more detail below.

The Patient Experience Diary (PED)

CLBP is a regional pain syndrome defined primarily by chronic pain in the low back; therefore, improvement in patient pain is an essential feature of any efficacious therapeutic intervention. Advances in both the technology and methodology of real-time data collection have enabled researchers to capture reliable and valid momentary data from subjects in the real world. [Stone A, S. S., Schwartz J, Broderick J, Hufford M, Patient compliance with paper and electronic diaries. Control Clin. Trials, 2003, 24(3), 182-99.] In this study, subjects are asked to provide information at up to five different times during the course of the day, including a morning report, evening report and on average, three random daily pain prompts.

To facilitate accurate and timely assessments of pain, an electronic diary system has been implemented for this study: Patient Experience Diary or PED (invivodata, inc., Pittsburgh, Pa.). The PED uses invivodata's proprietary software loaded on a personal digital assistant (PDA). The core of the PED data is the collection of subject self-reported data. In this study, the data are collected via entries made by subjects at relevant times into the PED. Specifically, the PED software enables subjects' pain assessments to be completed at a variety of times throughout the day, as required by the protocol.

The PED permits the collection of real-time, self-reported pain data by random report prompting multiple times daily, and also asks individuals to recall daily pain and weekly pain during the corresponding daily and weekly reports. The following table highlights key assessments implemented on the PED:

| Study | | |
|---|---|---|
| Phase(s) | Study Need | DIARY FUNCTION |
| Baseline and | Daily and “real- | Morning report |
| Treatment | time” pain data | Random prompts |
| | and daily mood | Evening report |
| | data | |
| | Weekly | Weekly report |
| | retrospective pain | |
| | and QOL data | |
| | Confirm | Self-initiated study medication |
| | medication | |
| | administration | |

The primary pain outcome variable is measured on the electronic diary. There are several additional pieces of pain information that are collected routinely during the clinic visits scheduled for Baseline Study Visit (BL2/Tx0), Tx6, and Tx12, such as pain self-report, psychophysical testing variables and standardized tenderness measures. Visual analog scale-based pain measurements are captured on a dedicated, daily and weekly pain recall case report form at study visits. These alternative pain assessment scales are evaluated as secondary variables, but do not substitute for data collected on the electronic diary.

In addition to pain ratings, assessments of mood and appetite are also recorded using the electronic diary. Subjects rate their mood and sedation nightly using a visual analog scale based on the Bond-Lader mood scale. [Bond, A. a. L., M., The use of analogue scales in rating subjective feelings. British Journal of Medical Psychology, 1974, 47, 211-218.] Subjects rate appetite on a weekly basis using a “drop-down” menu with the following choices: “increased”, “decreased”, or “no change”.

Training and Participant Usage. Following both a didactic and interactive training session at the Screening visit, participants are asked to use the PED to record symptoms daily for the duration of the 14-week study. The PED in this study prompts participants for several different types of input. In the morning, when participants first wake up, they report on their current pain level and their pain over the previous 24 hours. On multiple occasions throughout the day, random prompts requesting information about current level of pain are presented. Finally, at bedtime, another series of questions is presented, including a passive check of medication compliance. And on every 7th evening, participants are presented with the weekly report, which triggers another specific set of questions regarding their recall of pain and fatigue for the week. Each of these series of questions is designed to be easy and quick to complete, as minimizing burden on the participants has been carefully considered. All questions presented at all prompts are listed in the invivodata study coordinator manual.

To avoid interruptions to daily life, the random prompts may be suspended or delayed as needed for a period of 30 minutes up to 2 hours. The PED are pre-programmed with a standard wake period and evening report period, both substantial in duration to account for individual variations and habits. Following evening report, subjects place the PED in its dedicated modem for overnight data uploading and then awakening or activating PED the following morning within the programmed wake period. Because patient compliance is one of the major reasons to use the PED approach as compared to paper diaries, the electronic diary data are electronically time and date stamped when entries are made. There is no provision for the participant to make late entries.

Brief Pain Inventory: Clinical pain is also assessed using the Brief Pain Inventory (BPI). The BPI is a short, self-report measure that was originally developed for use in cancer patients to assess pain intensity and the impact of pain on the patient's life. [Tan G, J. M., Thornby J I, Shanti B F, Validation of the Brief Pain Inventory for chronic nonmalignant pain, J. Pain, 2004, 5(2)(March), 133-7; Keller S, B. C., Dodd S L, Schein J, Mendoza T R, Cleeland C S, Validity of the brief pain inventory for use in documenting the outcomes of patients with non-cancer pain, Clin. J. Pain, 2004, 20(5)(September), p. 309-18.] Recently, the BPI was validated for use in chronic, nonmalignant pain such as low back pain and arthritis with reliability and validity comparable to reports from the cancer literature and with internal consistency to support using the BPI as an outcome variable in treatment outcome studies. Tan, 2004. Patients are asked to rate their current pain intensity as well as their worst, least and average pain in the last 24 hours on a 0-10 rating scale (0=“no pain” and 10=“pain as bad as you can imagine”). Additionally, patients are asked to rate the extent that pain interferes with their life across 7 domains: general activity, walking, mood, sleep, work, relations with other persons, and enjoyment of life; interference is also rated on a 0-10 scale (0=“does not interfere” and 10=“completely interferes”).

Fatigue is assessed using the Multidimensional Fatigue Inventory (MFI). [Smets E M, G. B., Bonke B, De Haes J C, The Multidimensional Fatigue Inventory (MFI) psychometric qualities of an instrument to assess fatigue, Journal of Psychosomatic Research, 1995, 39(3)(April), 315-25. The MFI consists of 20 items that can be scored to produce 5 dimensions: general fatigue, physical fatigue, mental fatigue, reduced motivation, and reduced activity. This inventory has been validated in samples of cancer patients, medical students, army recruits and junior physicians. Internal consistency was demonstrated as acceptable for research and confirmatory factor analytic studies have supported the subscale structure of the inventory.

Sleep: The 12-item MOS Sleep Scale is a subscale of the larger Medical Outcomes Study test, and further, a subscale of a longer sleep scale of the same name. Despite its brevity it has been found to be comprehensive and empirically verified. Its questions are segregated into subscales addressing seven sleep domains (i.e. sleep disturbance, snoring, awaken short of breath or with headache, quantity of sleep, optimal sleep, sleep adequacy, and somnolence). Respondents indicate how often they have experienced each of the problems listed in the past 4 weeks on a six-point scale ranging from “none of the time” to “all of the time.” The MOS-Sleep Scale is a comprehensive battery of sleep questions whose dimensions have been empirically verified. [Stewart A L, W. J., Measuring Functioning and Well-Being, 1992, Duke University Press.]

General Functional Status is measured using the SF-36. [Ware J E J, S. C., The MOS 36-item short-form health survey (SF-36), I. Conceptual framework and item selection, Medical Care, 1992, 30(6)(June), 473-83.] The SF-36 is a brief, well-established, self-administered patient questionnaire for the assessment of health status. [Ware J E J, S. K., Kosinski M, Gandek B, SF-36 Health Survey Manual & Interprestation Guide, 2000, Lincoln: QualityMetric, Inc.] The SF-36 measures eight domains of health status: physical functioning, role limitations because of physical problems, bodily pain, general health perceptions, energy/vitality, social functioning, role limitations due to emotional problems, and mental health. A summary score for physical functional status (PCS) can be calculated by combining and weighting the various individual scales. [Ware J EJ, K. M., SF-36 Physical & Mental Health Summary Scales: A Manual for Users of Version 1. 2. 2001: Lincoln: QualityMetric, Inc.] The PCS score has been standardized to have a mean=50, SD=10 in the general US population. [Ware J, K. M., Keller MD, SF-36 physical and mental health summary scales: a user's manual, 1994, Boston: The Health Institute] Internal-consistency reliability for the PCS is r=0.91 with test-retest reliability being r=0.89.

Symptoms of anger and anxiety are assessed using the State-Trait Personality Inventory (STPI Form Y). [Spielberger, C., Preliminary manual for the State—Trait Personality Inventory (STPI). 2000.] The STPI is an 80-item self-report questionnaire with eight 10-item scales for measuring state and trait anxiety, anger, depression, and curiosity. For purposes of this study, a subset of 20-items are used that assesses trait anxiety and trait anger. The STPI possesses strong psychometric properties for the assessment of these mood symptoms given the items have been well validated as parts of larger instruments such as the State-Trait Anxiety Inventory and the State-Trait anger Inventory. [Spielberger C D, G. R., Lushene R, Manual for the State-Trait Anxiety Inventory: (STAI) “(Self-Evaluation Questionnaire),” 1979, Palo Alto: Consulting Psychologists Press.]

Brief Belief and Coping Assessment (BBCA). [Jensen M P, K. F., Lefebvre J C, Romano J M, Turner J A, One- and two-item measures of pain beliefs and coping strategies. Pain, 2003, 104(3)(August), 453-69.] The BBCA was developed as a means of assessing pain-related beliefs and coping strategies that are relevant to a biopsychosocial model of chronic pain. This instrument was derived from the subscales of five previously published belief and coping assessment tools: The Chronic Pain Coping Inventory (CPCI) [Jensen M P, T. J., Romano J M, Strom S E, The Chronic Pain Coping Inventory: development and preliminary validation, Pain, 1995, 60(2)(February), 203-16.], the Survey of Pain Attitudes (SOPA) [Jensen M P, T. J., Romano J M, Lawler B K, Relationship of pain-specific beliefs to chronic pain adjustment, Pain, 1994, 57(3)(June), p. 301-9.], the Pain Beliefs and Perceptions Inventory (PBAPI) [Williams, D. A., R. M., Geisser M E, Pain beliefs: Assessment and utility, Pain, 1994, 59(1)(October), p. 71-8.], the Arthritis self-efficacy scale (ASES) [Lorig K, C. R., Ung E, Shoor S, Holman H R, Development and evaluation of a scale to measure perceived self-efficacy in people with arthritis. Arthritis & Rheumatism, 1989. 32(1)(January), 37-44], and the Coping Strategies Questionnaire (CSQ) [Rosenstiel A K, K. F., The use of coping strategies in chronic low back pain patients. Relationship to patient characteristics and current adjustment, Pain, 1983, 17(1)(September), 33-44.] In order to briefly assess beliefs and coping, the BBCA validated one and two item versions of these existing scales: CPCI—guarding, resting, asking for assistance, relaxation, task persistence, exercise/stretch, seeking social support, coping self-statements; SOPA—Belief in pain control, disability, harm, emotion, medication, solicitude, medical cure; PBPI—pain as mystery, permanence, constancy, self-blame; CSQ—diverting attention, reinterpreting pain, catastrophizing, ignoring pain sensations, praying/hoping, coping self-statements, increased behavioral activity; ASES—self-efficacy to function, other symptoms, pain. Each of the two-item scales showed a strong association with the parent scale (R>=0.70) and 82% of the scales showed significant change with treatment.

Perceived Stress Scale (PSS) [Cohen S, K. T., Mermelstein R, A global measure of perceived stress, J. Health Soc. Behav., 1983, 24(4)(December), 385-96] is a 10-item psychological measure used to assess the perception of stress, or the degree that situations in one's life are determined to be stressful. The instrument asks patients to appraise their feelings and thoughts about how unpredictable, uncontrollable and overloaded their lives have been over the last month. Questions are general in nature and easy to understand.

Childhood Traumatic Events Scale (CTES). (Pennebaker J W, S. J., Disclosure of traumas and psychosomatic processes, Soc. Sci. Med., 1988. 26(3), 327-32.) The CTES questionnaire is composed of two forms. The first assesses childhood traumatic events that occurred prior to the age of 17. Domains include death of a close family member or friend, parental separation, physical abuse including sexual assault, serious illness, and other. For each question, the age of trauma, perceived intensity of the trauma, and whether or not confiding in others occurred is assessed. The second form is labeled Recent Traumatic Events Scale (RTES). It assesses essentially the same traumatic domains with the exception that the timeframe is within the last 3 years, parental separation is replaced with spouse or significant other separation, and a new category of job change is added. This instrument has been used to validate associations between traumatic events and current health problems.

The Multidimensional Scale of Perceived Social Support (MSPSS) [Zimet G D, D. N., Zimet S, Farley G K, The multidimensional scale of perceived social support, Journal of Personality Assessment, 1988, 52, 30-41] is a relatively brief, 12-item scale designed to assess three aspects of perceived social support: 1) support from friends, 2) support from family, and 3) support from significant others.

Activity Monitoring: The Actiwatch (Minimitter, Bend, Oreg.) is designed for long-term gross motor activity monitoring. The device is an omnidirectional sensor, which contains an accelerometer capable of sensing motion with a minimal resultant force of 0.01 g. The accelerometer integrates the degree and speed of motion and produces an electrical current; increased speed and motion result in an increase in voltage. This information is then stored as “Activity Counts” and can be downloaded later.

Subjects wear an activity monitor for the 2-week baseline symptom monitoring period: to assess sleep and overall activity; to correlate with PED symptom ratings; and to assess similarities with other chronic pain syndromes, such as fibromyalgia. Subjects are instructed to remove the watch only for protection against water immersion (i.e., showering, swimming, etc.)

Psychophysical Assessment of Pain: In this study, a range of pain measures from highly subjective to objective via psychophysical tests of evoked pain sensitivity: a discrete, ascending pressure pain test; and a discrete, multiple random staircase pressure pain test are performed. Criteria for the diagnosis of chronic pain syndromes include both the presence of ongoing clinical (i.e. spontaneous) pain and an evoked-pain abnormality.

Assessment of Tenderness. This study employs the manual tender point exam as a means of assessing experimental tenderness in a CLBP population. The manual tender point examination is performed using standardized methodology [Smythe, H., Examination for tenderness: learning to use 4 kg force, Journal of Rheumatology, 1998, 25(1)(January), 149-51], with manual pressure of approximately four kilograms applied to 11 bilateral musculotendinous points. Participants indicate if they experience tenderness at a particular site, thus giving a dichotomous “yes/no” response at each point.

Evoked Pressure Testing. At the Baseline Study Visit (BL2/Tx0), Tx6, and Tx12, the planned study uses the methods that apply discrete, pressure stimuli that have been developed and used in previous studies. [Giesecke, T., et al., Evidence of augmented central pain processing in idiopathic chronic low back pain. Arthritis Rheum., 2004, 50(2), 613-23; Gracely R H, L. L., Walter D J, Dubner R, A multiple random staircase method of psychophysical pain assessment, Pain, 1988, 32(1)(January), 55-63; Gracely R H, P. F., Wolf J M; Clauw D J., Functional magnetic resonance imaging evidence of augmented pain processing in fibromyalgia, Arthritis Rheum., 2002. 46(5)(May), 1333-43]. In these methods, a number of stimulus intensities are chosen with sufficiently close spacing to be confused, resulting in independent judgments of sensation rather than judgments of stimulus identification (e.g., “that is the third one from the bottom”). An “adaptive” method further reduces the influence of extraneous psychological factors by automatically adjusting stimulus intensity to each subject's subjective range, and by minimizing external cues of baseline sensitivity (e.g., expressive or stoical) or of altered sensitivity. [Petzke F, G. R., Park K M, Ambrose K, Clauw D J, What do tender points measure? Influence of distress on 4 measures of tenderness. J. Rheumatol., 2003, 30(3)(March), 567-74.]

Pressure stimuli are delivered by psychophysical methods. Since ascending methods represent the most widely used methods of pain threshold assessments in clinical practice, the following pain testing paradigm was developed as a sophisticated method to compare a traditional tender point count or dolorimeter exam to both ascending and random pressure testing paradigms that assess both threshold and suprathreshold pressure pain sensitivity.

The random pressure-pain testing paradigms have been shown to be relatively immune to the psychophysical biases that influence the clinical ascending methods. This study assesses pressure pain sensitivity at the thumbnails using a device that consists of a plastic housing and piston driving a hard rubber 1 cm2 probe. The piston can be driven by either the original manual hydraulic system using calibrated weights, or by a computerized pneumatic system. Each system is driven by the same software logic. After positioning the device on the left thumb, subjects receive an ascending series of discrete 3 second-duration pressure stimuli beginning at 0.5 kg and increasing in 0.5 kg steps to a maximum of 10 kg. (In terms of pressure delivered through the stimulating surface, the first stimulus is 49 kPa and the stimuli are increased in 49 kPa steps to a maximum pressure of 980 kPa). Subjects rate the intensity of the evoked sensations using the sensory intensity Box scale, a combined analog descriptor scale that superimposes verbal descriptors, spaced according to ratio-scale values, on a 0-20 graphical numerical category scale.

This series is repeated and the results are used to select the initial stimuli for the Multiple Random Staircase (MRS) paradigm [Petzke F, G. R., Park K M, Ambrose K, Clauw D J, What do tender points measure? Influence of distress on 4 measures of tenderness, J. Rheumatol., 2003, 30(3)(March), 567-74], which uses the same Box scale. The algorithm alternates between three individual staircases that titrate stimulus intensity to produce pain responses at pain threshold (scale value 0.5), mild to moderate pain (scale value 9.5), and strong to slightly intense pain (scale value 13.5). For each staircase, a response below the desired level increases the intensity of the stimulus next chosen for the stimulus and a response above the desired level lowers the intensity of the next stimulus delivered by that staircase. The amount the stimulus is changed is also controlled by the software. This increment initially is large (0.8 kg) and is reduced if the staircase quickly changes direction, and increased if the staircase continuously ascends or descends for a set (usually 4) number of trials. In the present application of this method 5-second duration pressure stimuli are delivered once every 30 s and 24 stimuli are delivered for each of the three staircases for a total of 72 stimuli delivered over 36 min.

Salivary Cortisol

Salivary cortisol is collected during the baseline symptom monitoring phase to assess similarities between CLBP and other chronic pain syndromes (i.e., fibromyalgia, irritable bowel syndrome, temporomandibular disorder, etc).

Heart Rate Variability: Information regarding autonomic nervous system function in response to stress is obtained and assessed by monitoring heart rate variability (HRV).

HRV information is obtained via a brief (<60 minutes) Holter monitor recording during each subject's Baseline Study Visit (BL2/Tx0). Individuals remain supine for first 5 minutes of this time period, prior to any study related testing. For the remaining time period, the Holter monitor records a continuous electrocardiogram (ECG) of the heart's electrical activity, which is then uploaded to a centralized computer database and sent out for batch analysis.

Functional Imaging (e.g. fMRI): Functional MRI is based on the principle that the MRI signal changes in response to the magnetic character of the intravascular contents. Because deoxygenated hemoglobin is more magnetic than oxygenated hemoglobin, it acts as an endogenous intravascular paramagnetic contrast agent. Variations in blood oxygenation therefore affect the MR signal intensity, hence the term Blood Oxygenation Level Dependent (BOLD) contrast. It is this phenomenon on which most fMRI is based. During increased neural activity, there is an elevation of cerebral blood flow greater than required to support local oxygen consumption. As a result of this discrepancy, the relative local concentration of deoxyhemoglobin decreases. This decrease reduces the magnetic suppression of the BOLD signal from nearby tissue. The end result is increased signal intensity, which, as in positron emission tomography (PET), allows estimation of task-related neural activation when compared to a baseline image. fMRI has been used to investigate the processes underlying human motor control, visual perception and cognition.

In this study, fMRI and evoked pressure pain stimuli are used to determine whether CLBP patients exhibit similar activations as fibromyalgia patients and, thus, exhibit similar central pain augmentation.

fMRI Methods: A subset of subjects undergo both pre- and post-intervention scanning sessions (approx. 24 hrs following evoked pressure testing at Baseline Study Visit [BL2/Tx0] and Tx 12). Each fMRI evaluation consists of a 1.5-2 hr session that includes an anatomical MRI of the head and multiple functional scans in which subjects receive painful pressures applied to the left thumb. One type of scan administers a series of 10 stimuli to the left thumb with the same temporal parameters (30 s “on,” 30 s “off”) used in a previous study of fibromyalgia. [Gracely R H, P. F., Wolf J M, Clauw D J., Functional magnetic resonance imaging evidence of augmented pain processing in fibromyalgia. Arthritis Rheum, 2002. 46(5)(May), 1333-43.] Stimulus intensity is constant during the scan, and is adjusted to deliver constant ratings of moderate to slightly-intense pain on the intensity Box scale, and in the case of control subjects, also to match the level of stimulation delivered to the patients. Another type of scan varies stimulus intensities during the scan. This procedure follows the same method used in other preliminary experiments: four stimulus levels (one innocuous, three painful) are presented 3 times each in random sequence. Duration of stimulus on and off conditions are 25 s. Stimulus intensities are individually determined for each subject, based on baseline psychophysical results, to produce pain sensations corresponding to mild, moderate and slightly intense on the intensity Box scale.

Anatomical images of the head are examined subjectively by research personnel and objectively warped to a normal brain for group analysis. Both of these procedures are designed to identify brains that cannot be conformed to a standard for group analyses.

Treatment Strategy: After initial screening, each subject returns to the clinic to begin the baseline period (BL0 to BL2). Each subject enters a two-week baseline observation phase. At the start of this two-week period (BL0), each subject is issued a PED device. Each patient is trained to operate the PED. During the baseline period, subjects become comfortable with the PED technology and establish their baseline pain and disease activity.

The interval between Screening/BL0 and BL2 is at least 10 days, but not more than 21 days. If more than 14 days of baseline pain data are collected on the PED, values from the last 14 days are averaged to obtain the patient's baseline pain score.

Mirtazapine is administered as over-encapsulated 15 mg tablets. Zonisamide is administered as over-encapsulated 100 mg capsules. Amitriptyline is administered as over-encapsulated 10 and 25 mg tablets. Diphenhydramine is administered as an over-encapsulated capsule containing 25 mg of active placebo. All capsules are identically over-encapsulated in order to maintain blinding of patients, staff and investigators. All capsules are identical in appearance by means of over-encapsulation utilizing size #0 hard, Swedish orange, gelatin capsules.

Randomization: Up to 160 men and women are randomized into four separate study groups, each of which receives either placebo or drug once per day, in a 1:1:1:1 ratio. The first group is the placebo (P) arm, in which each subject receives diphenhydramine as an active placebo. The second group is the control (C) arm, in which each subject receives amitriptyline as an active control. The third group is trial level 1 (T1), in which each subject receives 15 mg mirtazapine and 100 mg zonisamide per day. The fourth group is trial level 2 (T2), in which each subject receives 30 mg mirtazapine and 100 mg zonisamide per day.

Randomization Procedures: Patients are randomly assigned to one of four treatment groups. They are assigned a Patient Number in chronological order at the time of screening. The randomization ratio is 1:1:1:1. A randomization list is generated and provided to the drug packaging facility. The facility packages the drug according to this randomization list. The pharmacist at the study site is provided with a list that relates each subject number to a particular group. Each group is designated to the pharmacist as A, B, C or D. The pharmacist pulls the appropriate dose level of drug for each patient based on the list. The pharmacist is unaware of which drug corresponds to each letter.

Once randomized, subjects enter the dose titration phase of the study. Patients who do not successfully complete the baseline period with respect to compliance with the PED are not randomized, and are terminated from the study.

Dose Titration Phase [Weeks TX0-TX4]: For blinding purposes, placebo (P) as well as active subjects (C, T1, T2) undergo dose escalation, and identical-appearing capsules are used by all subjects during the trial. Subjects, investigators, other site staff, and the sponsor remain blinded to patients' treatment randomization.

Dose Titration Interval Timing: All subjects undergo a four week dose titration, as detailed below. Subjects may choose to stop escalating at any particular dose level, based on tolerability and perceived efficacy.

Active Placebo (diphenhydramine)
Week 1: Subjects receive one 25 mg capsule containing diphenhydramine and 3 inert capsules.
Week 2: Patients receive one 25 mg capsule containing diphenhydramine and 3 inert capsules.
Week 3: Patients receive one 25 mg capsule containing diphenhydramine and 3 inert capsules.
Week 4: Patients receive two 25 mg capsules containing diphenhydramine and 2 inert capsules.

Active Control (Amitriptyline 25 mg)
Week 1: Patients receive one 10 mg amitriptyline capsule and 3 inert capsules.
Week 2: Patients receive two 10 mg amitriptyline capsules and 2 inert capsules.
Week 3: Patients receive three 10 mg amitriptyline capsules and 1 inert capsule.
Week 4: Patients receive two 25 mg amitriptyline capsules and 2 inert capsules.

Trial Dose 1: Mirtazapine/Zonisamide (T1: Mirtazapine 15 mg Zonisamide 100 mg)
Week 1: Patients receive one 7.5 mg mirtazapine capsule, one 50 mg zonisamide capsule and 2 inert capsules.
Week 2: Patients receive one 15 mg mirtazapine capsule, one 100 mg zonisamide capsule and 2 inert capsules.
Week 3: Patients receive one 15 mg mirtazapine capsule, one 100 mg zonisamide capsule and 2 inert capsules.
Week 4: Patients receive one 15 mg mirtazapine capsule, and one 100 mg zonisamide capsule and 2 inert capsules.

Trial Dose 2: Mirtazapine/Zonisamide (T2: Mirtazapine 30 mg Zonisamide 200 mg)
Week 1: Patients receive one 7.5 mg mirtazapine capsule, one 50 mg zonisamide capsule and 2 inert capsules.
Week 2: Patients receive one 15 mg mirtazapine capsule, one 100 mg zonisamide capsule and 2 inert capsules.
Week 3: Patients receive two 15 mg mirtazapine capsules, two 100 mg zonisamide capsules and 2 inert capsules.
Week 4: Patients receive two 15 mg mirtazapine capsules, two 100 mg zonisamide capsules.

A. Treatment Phase [Weeks TX4-TX12/ET]

Once the patient has completed the dose titration phase, a minimum of 7 weeks of treatment (TX4 to TX12) begins at a steady dose level. Once a patient has entered the stable dose treatment phase, the patient is expected to complete daily and weekly PED assessments. Clinic visits take place at Tx2, Tx4, Tx6 and Tx12; all subjects receive a phone call or email during dose titration and at TX9 to assess tolerability and side effect issues.

Daily and Weekly Home Assessments: The PED device prompts the patient several times throughout the day to record various aspects of their current status. The morning report requests information about patients' level of pain during the previous 24 hours. The patient is also prompted several times during the day in a semi-random fashion for information regarding their current level of pain. An evening prompt (around bedtime) requests information about patients' current level of pain, pain medication use, menstruation, depression and anxiety. There is an additional weekly report on every 7th day that captures information about the patient's mood, ability to do usual daily activities, walking ability, normal work, and relations with other people, sleep and enjoyment of life.

Change in electronic diary pain from a baseline to the endpoint at week 12 is measured. The mean percentage reduction in pain for actively treated patients is compared to that of placebo-treated patients. Pain reduction is calculated by comparing the average of the last 2 weeks (weeks 11 and 12) daily 24 hour recall pain reports for the 2 week baseline average.

B. Dose Tapering Phase [Weeks TX12-TX16]

Once a patient completes the treatment phase, a 2-week dose taper begins. Patients receive the final supply of study drug at a dose that is half the strength of their tolerated stable dose. At Week Tx14, all patients receive a phone call or email to assess side effects with dose reduction and stoppage. At Week Tx16, patients return for a final study visit to assess vital signs, side effects or adverse events with discontinuation of drug, questionnaires and study closeout activities.

Subjects receive the first 2-week supply of study drug at study visit BL2/Tx0. Subjects are contacted on weeks they are not required to come to the clinic as described below. Clinic visits are conducted for purposes of drug dispensing and/or data collection, also as described below:
Week TX 1 Phone/email contact: All study subjects will receive a phone call or email at the beginning of Week 1. The purpose of this contact is to determine whether or not the subject is having difficulty with the upward dose escalation of the study drug. Subjects are similarly queried for adverse events and concomitant medication usage.
Week Tx2 Clinic Visit (+/−7 days): At week Tx4 of the Treatment Period (+/−7 days), the patient completes dose escalation, and visits the clinic office to obtain two weeks of drug supply. During this clinic visit, information concerning adverse events, sleep and pain, as well as psychological and functional status, is collected.
Week TX 3 Phone/email contact: All study subjects receive a phone call or email at the beginning of Week 3. The purpose of this contact is to determine whether or not the subject is having difficulty with the upward dose escalation of the study drug. Subjects are similarly queried for adverse events and concomitant medication usage.
Week Tx4 Clinic Visit (+/−7 days): At week Tx4 of the Treatment Period (+/−7 days), the patient completes dose escalation, and visits the clinic office to obtain two weeks of drug supply. During this clinic visit, information concerning adverse events, sleep and pain, as well as psychological and functional status, is collected.
Week Tx6 Clinic Visit (+/−7 days): At weeks Tx6 of the Treatment Period (+/−7 days), the patients return to the clinic to obtain their next six weeks of drug supply. During this clinic visit, information concerning adverse events, sleep and pain, as well as psychological and functional status, is collected.
Week 9 Phone/email Contact: All study subjects receive a phone call or email at the beginning of Week 9. The purpose of this contact is to query subjects regarding adverse events and concomitant medication usage.
Week TX12 Clinic Visit: At weeks TX12 of the Treatment Period (+/−7 days), the patients return to the clinic for their final study visit. During this clinic visit, information concerning adverse events, sleep and pain, as well as psychological and functional status, is collected. Additionally, patients will obtain the final two weeks of drug supply.
Week TX14 Phone/email Contact: All study subjects receive a phone call or email at the beginning of Week 14. The purpose of this contact is to determine whether or not the subject is having difficulty with tapering off of the study drug. Subjects are similarly queried for adverse events and concomitant medication usage.
Week Tx16 Clinic Visit (+/−7 days): At week Tx16 of the Dose Tapering Period (+/−7 days), the patient completes dose therapy 2 weeks prior to this visit. During this clinic visit, information concerning adverse events, sleep and pain, as well as psychological and functional status, are collected. Study termination activities will occur at this visit.

Early Termination Visit: The termination form is completed at Week TX12 or at any visit at which a randomized patient is terminated from the study. If a patient exits the protocol prior to Week Tx 12, the assessments indicated for Week TX12, as outlined in Study Parameters section VII., are completed. These patients are classified as early terminations.

Clinical Endpoints

Efficacy of the combination of the combination of mirtazapine and zonisamide is assessed using the following methods:

Self-reporting questionnaires

Electronic patient experience diary (PED)

Activity monitoring

Psychophysical assessment of pain

Functional imaging (e.g. functional MRI).

Those skilled in the art will recognize, or be able to ascertain using no more than routine experimentation, many equivalents to the specific embodiments of the invention described herein. Such equivalents are intended to be encompassed by the following claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of reducing the incidence or severity of one or more side effects associated with the administration of a 5HT2/5HT3 antagonist/alpha-2 antagonist in the treatment of a disorder in an individual comprising administering an effective amount of a second compound selected from the group consisting of a dopamine-releasing compound, anticonvulsant, and dopamine/norepinephrine reuptake inhibitor in combination with the 5HT2/5HT3 antagonist/alpha-2 antagonist.

2. The method of claim 1 wherein the 5HT2/5HT3 antagonist/alpha-2 antagonist and the second compound are administered simultaneously.

3. The method of claim 1 wherein the second compound is administered or released from a formulation after the 5HT2/5HT3 antagonist/alpha-2 antagonist.

4. The method of claim 1, wherein the 5HT2/5HT3 antagonist/alpha-2 antagonist is selected from the group consisting of setiptiline and mirtazapine.

5. The method of claim 1, wherein the second compound is a dopamine-releasing compound selected from the group consisting of amantadine, racemic rimantadine, (R)-rimantadine, (S)-rimantadine, and combinations of thereof.

6. The method of claim 1, wherein the second compound is an anticonvulsant selected from the group consisting of zonisamide and topiramate.

7. The method of claim 1, wherein the second compound is a dopamine/norepinephrine reuptake inhibitor selected from the group consisting of bupropion, racemic sibutramine, (+)-sibutramine, (−)-sibutramine, racemic didesmethylsibutramine, (+)-didesmethylsibutramine, (−)-didesmethylsibutramine, racemic desmethylsibutramine, (+)-desmethylsibutramine, and (−)-desmethylsibutramine.

8. The method of claim 1 wherein the side effects are selected from the group consisting of excessive daytime sleepiness and weight gain.

9. The method of claim 1 wherein the disorder is selected from the group consisting of depression, schizophrenia, anxiety disorders, sleep-related breathing disorders, insomnia, migraine headache, chronic tension-type headache, hot flashes, chronic lower back pain, neuropathic pain and functional somatic syndromes.

10. The method of claim 9 wherein the disorder is an anxiety disorder selected from the group consisting of generalized anxiety disorder, panic disorder, phobias, and post-traumatic stress disorder.

11. The method of claim 9 wherein the disorder is a sleep-related breathing disorder selected from the group consisting of sleep apnea, sleep hypopnea, upper airway resistance syndrome, and snoring.

12. The method of claim 9 wherein the disorder is a functional somatic syndrome selected from the group consisting of fibromyalgia syndrome, chronic fatigue syndrome, and irritable bowel syndrome.

13. The method of claim 1 wherein the second compound is a dopamine-releasing compound administered in an amount between 50 to 400 mg/day and the amount of 5HT2/5HT3 antagonist/alpha-2 antagonist administered is 5 to 200 mg/day.

14. The method of claim 1 wherein the second compound is an anticonvulsant administered in an amount between 10 to 600 mg/day and the amount of 5HT2/5HT3 antagonist/alpha-2 antagonist administered is 5 to 200 mg/day.

15. The method of claim 1 wherein the second compound is a dopamine/norepinephrine reuptake inhibitor administered in an amount between 50 and 400 mg/day and the amount of 5HT2/5HT3 antagonist/alpha-2 antagonist administered is between 5 and 200 mg/day.

16. The method of claim 1 wherein the formulation is administered in an escalating dosage.

17. The method of claim 1 wherein the formulation is administered for a period of time, followed by a washout period, and then administered again.

18. A formulation comprising an effective amount of a 5HT2/5HT3 antagonist/alpha-2 antagonist for the treatment of a disorder and an effective amount of a second compound selected from the group consisting of a dopamine-releasing compound, anticonvulsant, and dopamine/norepinephrine reuptake inhibitor to reduce the incidence or severity of one or more side effects associated with the 5HT2/5HT3 antagonist/alpha-2 antagonist in an individual.

19. The formulation of claim 18 wherein the side effect is selected from the group consisting of weight gain and excessive daytime sleepiness.

20. The formulation of claim 18 wherein the disorder is selected from the group consisting of depression, schizophrenia, anxiety disorders, sleep-related breathing disorders, insomnia, migraine headache, chronic tension-type headache, hot flashes, chronic lower back pain, neuropathic pain and functional somatic syndromes.

21. The formulation of claim 20 wherein the disorder is an anxiety disorder selected from the group consisting of generalized anxiety disorder, panic disorder, phobias, and post-traumatic stress disorder.

22. The formulation of claim 20 wherein the disorder is a sleep-related breathing disorder selected from the group consisting of sleep apnea, sleep hypopnea, upper airway resistance syndrome, and snoring.

23. The formulation of claim 20 wherein the disorder is a functional somatic syndrome selected from the group consisting of fibromyalgia syndrome, chronic fatigue syndrome, and irritable bowel syndrome.

24. The formulation of claim 18 wherein the 5HT2/5HT3 antagonist/alpha-2 antagonist is in an immediate release formulation and the second compound is in a delayed release formulation.

25. The formulation of claim 18 wherein the second compound is released at least six hours after the 5HT2/5HT3 antagonist/alpha-2 antagonist.

26. The formulation of claim 18, wherein the 5HT2/5HT3 antagonist/alpha-2 antagonist is selected from the group consisting of mirtazapine and setiptiline.

27. The formulation of claim 18, wherein the second compound is a dopamine-releasing compound selected from the group consisting of amantadine, racemic rimantadine, (R)-rimantadine, (S)-rimantadine, and combinations of thereof.

28. The formulation of claim 18, wherein the second compound is an anticonvulsant selected from the group consisting of zonisamide and topiramate.

29. The formulation of claim 18, wherein the second compound is a dopamine/norepinephrine reuptake inhibitor selected from the group consisting of bupropion, racemic sibutramine, (+)-sibutramine, (−)-sibutramine, racemic didesmethylsibutramine, (+)-didesmethylsibutramine, (−)-didesmethylsibutramine, racemic desmethylsibutramine, (+)-desmethylsibutramine, and (−)-desmethylsibutramine.

30. The formulation of claim 18 comprising 50 to 400 mg dopamine-releasing compound and 5 to 200 mg 5HT2/5HT3 antagonist/alpha-2 antagonist.

31. The formulation of claim 18 comprising 10 to 600 mg anticonvulsant and 5 to 200 mg 5HT2/5HT3 antagonist/alpha-2 antagonist.

32. The formulation of claim 18 comprising between 50 and 400 mg dopamine/norepinephrine reuptake inhibitor and between 5 and 200 mg 5HT2/5HT3 antagonist/alpha-2 antagonist.

33. The formulation of claim 18 for oral administration.

34. The formulation of claim 18 as a liquid dispersion or solution.

35. The formulation of claim 18 as a tablet, capsule, powder, microparticles, granules, or enteric coated formulation.

36. A method of treating chronic lower back pain in a patient, comprising administering to the patient a therapeutically effective combination of mirtazapine and zonisamide.

37. The method of claim 36, wherein the combination comprises about 7.5 to about 200 mg of mirtazapine per day.

38. The method of claim 37, wherein the combination comprises about 15 to about 45 mg of mirtazapine per day.

39. The method of claim 38, wherein the combination comprises about 15 or about 30 mg of mirtazapine per day.

40. The method of claim 36-39, wherein the combination comprises about 10 to about 600 mg of zonisamide per day.

41. The method of claim 40, wherein the combination comprises about 50 to about 400 mg of zonisamide per day.

42. The method of claim 41, wherein the combination comprises about 100 or about 200 mg of zonisamide per day.

43. The method of claim 36, wherein the amount of zonisamide is sufficient to prevent or reduce excessive daytime sleepiness in the patient.

44. The method of claim 36, wherein the amount of zonisamide is sufficient to prevent or reduce drowsiness in the patient.

45. The method of claim 36, wherein the amount of zonisamide is sufficient to prevent or reduce weight gain in the patient.

46. A method of treating chronic lower back pain in a patient, comprising administering to the patient a therapeutically effective combination of setiptiline and zonisamide.

47. The method of claim 46, wherein the combination comprises about 5 to about 50 mg of setiptiline per day.

48. The method of claim 47, wherein the combination comprises about 5 to about 20 mg of setiptiline per day.

49. The method of claim 48, wherein the combination comprises about 5 or about 10 mg of setiptiline per day.

50. The method of claim 46-49, wherein the combination comprises about 10 to about 600 mg of zonisamide per day.

51. The method of claim 50, wherein the combination comprises about 50 to about 400 mg of zonisamide per day.

52. The method of claim 51, wherein the combination comprises about 100 or about 200 mg of zonisamide per day.

53. The method of claim 46, wherein the amount of zonisamide is sufficient to prevent or reduce excessive daytime sleepiness in the patient.

54. The method of claim 46, wherein the amount of zonisamide is sufficient to prevent or reduce drowsiness in the patient.

55. The method of claim 46, wherein the amount of zonisamide is sufficient to prevent or reduce weight gain in the patient.
